(19) United States
(12) Patent Application Publication
(10) Pub. No.: US 2016/0117937 A1
(43) Pub. Date: Apr. 28, 2016

(54) SYSTEM AND METHOD FOR PROVIDING BIOMETRIC AND CONTEXT BASED MESSAGING

(71) Applicant: Bloom Technologies NV, Diepenbeek (BE)

(72) Inventors: Julien Penders, Liege (BE); Marco Altini, Eindhoven (NL); Eric Dy, San Francisco, CA (US); Torfinn Berset, Veldhoven (NL)

(21) Appl. No.: 14/524,640

(22) Filed: Oct. 27, 2014

(51) Int. Cl. G09B 5/00; H04L 29/08; G06F 17/30; G09B 19/00

(52) U.S. Cl. CPC G09B 5/00; G09B 19/00; H04L 67/10; G06F 17/30867

(57) ABSTRACT

A system suitable for generating messages based on biometric and contextual data and methods for making and using the same. One or more sensors can be used to measure biometric and/or contextual data. The measured data are analyzed using behavior analytics to capture behavior of a selected user. The behavior is analyzed with messaging analytics to generate personalized messages related to the behavior. The message preferably is personalized at at least one level, such as time, place and format of message delivery, content of the message, and tone of the message. The message personalization can further adapt to changes in the user's behavior and/or preferences. Thereby, the method and system advantageously can provide biometric and context based messaging for motivating healthy behavior.

### FIELD

The disclosed embodiments relate generally to the fields of behavior science and mobile health, and, more specifically, to a method and a system for biometric and context based messaging to motivate behavior change.

### BACKGROUND

The cost of healthcare delivery is steadily on an upward trend. US health care spending is estimated at approximately 17% of the Gross Domestic Product (GDP). This upward trend is expected to continue, with projections that the health share of the GDP reaches 19.5% by 2017. Health care spending in other OECD countries is projected to consume up to 16% of GDP. It has been established that ¾ of the healthcare costs are directly attributable to lifestyle related chronic diseases. In the same way, lifestyle behaviors account for 40% of mortality in industrialized countries. Adherence to key lifestyle behaviors has been shown to reduce coronary events and cardiovascular events. Lifestyle behaviors are of particular importance for women of childbearing age, as it has been shown that lifestyle modifications before and during pregnancy have the potential to reduce the risk of maternal and fetal complications and chronic diseases. Furthermore, pregnancy has been identified as a window of opportunity for long-term lifestyle changes.

The science of health behavior change aims at understanding what motivates people to change their behavior and maintain healthier lifestyle. It provides a theoretical framework to study, establish and validate behavior change interventions. Digital and mobile technologies, and the emergence and mass adoption of the smartphone in particular, are revolutionizing healthcare by connecting patients and doctors in ways never possible before and providing an ubiquitous platform to collect, exchange and visualize health and behavior data. Smartphones now also integrate a multitude of sensors that can be used to gather information about a person's health and behavior. Wearable sensors complement smart phones and allow tracking biometric data continuously and 24/7, opening new opportunities in modeling health and behavior.

In recent years researchers have started studying the use of digital and mobile technologies in behavior science. In early 2000, S. Intille, Ubiquitous Computing Technology for Just-in-Time Motivation of Behavior Change, Stud Health Technol Inform. 2004; 107(Pt 2):1434-7, introduced the concept of just-in-time motivation using sensing technology. A. Salah et al., Human Behavior Understanding for Inducing Behavioral Change: Application Perspectives, Human Behavior Understanding, Lecture Notes in Computer Science, Volume 7065, 2011, pp 1-15, describes how pervasive sensing can be used to understand human behavior and drive behavior change. N. Lathia et al., Smart phones for large-scale behavior change interventions, Proceedings of IEEE Pervasive Computing, 2013, introduces the concept of Digital Behavioral Change Induction to denote the use of digital technologies in behavior change.

Behavior change interventions reported to date have had varying levels of success however. In particular the use of messaging to motivate behavior change can be inefficient if the messages are not delivered in a proper way. The main problem of existing messaging approaches to behavior change is the efficacy of the message-based behavior change interventions. More specifically, there are four main challenges in defining an efficient strategy to behavior change:

Message should come at the right time: the message should be delivered at a time that maximize the chance of the user taking action;

Message should come at the right place: the message should be delivered at a place that allows the user to take action;

Message should be relevant to the current situation: the message should be prompted in reaction to specific behaviors exhibited by the user, and based on the knowledge of the user's health and behavior, as opposed to a pre-defined sequence of messaging that will lead to messages that may not be relevant for the user in the current situation; and

Message should be personalized and adaptive: the message should be delivered in a way that is specific and customized to the user (personalized), in terms of its content, tone, format and platform of delivery, and in a way that dynamically adapts as the user is undergoing behavior change (adaptive).

S. Intille reported the first two challenges and highlighted the importance of using a simple message that is easy to understand, and that is delivered in a non-annoying way.

A. Salah et al. reviewed the concept of Human Behavior Understanding (HBU) in the context of behavioral change, which they define as “pattern recognition and modeling techniques to automatically interpret complex behavioral patterns generated when humans interact with machines or other humans.” Pervasive sensing technologies such as smart phones and wearable sensors are typically used to analyze the behaviors. The authors report that human behavior understanding can be used at several levels of the behavior change induction process, including:

Positioning, in which the source of the information is using HBU to position the recipient, and selects appropriate message;

Message, in which the result of HBU is part of the message itself

Evaluation, in which HBU is used to track the progress of the recipient after he received the message; and

Prediction, in which HBU is used to predict future behavior, to allow the system to adapt and possibly preempt.

Combining these different levels, HBU has the potential to improve the channel, the message and the source of a certain messages aimed at behavior change. The authors do not provide any information on the actual implementation of HBU in the behavior change approach, but merely provide a few examples of applications where such an approach could be beneficial.

Lathia et al. reported an approach to Digital Behavior Change Intervention (DBCI) using smart phones, and based on three key components: monitor behavior, learn and infer behavior, deliver target behavior change. With their approach, Lathia et al. address the challenges of delivering information at the right time and, to a certain extent, the personalization of the behavior change intervention. The personalization is however limited, and mainly targeted to the personalization of the user interface.

The approaches by Intille et al. and Lathia et al., as well as other well-known approaches available to analyze sensor data and extract physical and physiological information regarding the measured body, omit the fact that sensor data also carries information about context, biometric and individual characteristics of the user that should be used in combination with physical and physiological information to model behavior. Existing methods also fail to exploit the fact that behavior models can be further analyzed to drive the creation of messages that are directly related to the context and modeled behavior, and that are delivered in a format and a way that is the most suitable for the user. Furthermore, existing methods fail to capture that, when combined with the knowledge of a target behavior change, these messages can be used to effectively drive behavior change.

In view of the foregoing, a need exists for an improved system and method for delivering relevant, personalized and adaptive messaging at the right time and the right place, and thus increasing the efficiency of messaging-based behavior change.

### BRIEF DESCRIPTION OF THE DRAWINGS

FIG. 1 is an exemplary top-level flow diagram illustrating an embodiment of a messaging method suitable for biometric and context based messaging, wherein the method includes measuring sensor data, performing behavior analytics and performing messaging analytics.

FIG. 2 is an exemplary flow diagram illustrating an alternative embodiment of the messaging method of FIG. 1, wherein the messaging method further includes storing messages in a message database.

FIG. 3 is an exemplary flow diagram illustrating an alternative embodiment of the messaging method of FIG. 2, wherein the messaging method further includes delivering messages to the user.

FIG. 4A is an exemplary detail flow diagram illustrating another alternative embodiment of the messaging method of FIG. 1, wherein measuring sensor data includes receiving raw sensor signals and acquiring raw sensor signals.

FIG. 4B is an exemplary detail flow diagram illustrating yet another alternative embodiment of the messaging method of FIG. 4A, wherein acquiring raw sensor signals includes amplifying raw sensor signals, conditioning raw sensor signals and converting raw sensor signals from analog to digital.

FIG. 5A is an exemplary detail flow diagram illustrating an alternative embodiment of the messaging method of FIG. 4A, wherein measuring sensor data further includes pre-processing raw sensor signals.

FIG. 5B is an exemplary detail flow diagram illustrating an alternative embodiment of the messaging method of FIG. 5A, wherein pre-processing raw sensor signals includes filtering raw sensor signals and translating signals to physical, physiological and/or environment signals.

FIG. 6A is an exemplary detail flow diagram illustrating yet another alternative embodiment of the messaging method of FIG. 1, wherein performing behavior analytics includes processing biometric data, processing contextual data, extracting behavior markers and modeling behavior.

FIG. 6B is an exemplary detail flow diagram illustrating an alternative embodiment of the messaging method of FIG. 6A, wherein the method further includes personalizing behavior analytics.

FIG. 7A is an exemplary detail flow diagram illustrating yet another alternative embodiment of the messaging method of FIG. 1, wherein performing message analytics includes interpreting behavior models and generating messages.

FIG. 7B is an exemplary detail flow diagram illustrating an alternative embodiment of the messaging method of FIG. 7A, wherein the method further includes customizing messages.

FIG. 7C is an exemplary detail flow diagram illustrating an alternative embodiment of the messaging method of FIG. 7B, wherein the method further includes personalizing message analytics.

FIG. 8A is an exemplary flow diagram illustrating another alternative embodiment of the messaging method of FIG. 7A, FIG. 7B or FIG. 7C, wherein generating messages includes scheduling messages, querying message database with message attributes, and reading messages back from message database.

FIG. 8B is an exemplary detail flow diagram illustrating yet another alternative embodiment of the messaging method of FIG. 7A, FIG. 7B or FIG. 7C, wherein generating messages further includes filtering messages.

FIG. 8C is an exemplary detail flow diagram illustrating yet another alternative embodiment of the messaging method of FIG. 7A, FIG. 7B or FIG. 7C, wherein generating messages includes scheduling messages and creating messages based on the behavior models.

FIG. 9A is an exemplary top-level block diagram illustrating an embodiment of a system for biometric and context based messaging, which includes a measurement system, an analytics system and an information system.

FIG. 9B is an exemplary block diagram illustrating an alternative embodiment of the messaging system of FIG. 9A, wherein the system includes a user interface system to communicate with the user.

FIG. 9C is an exemplary block diagram illustrating another alternative embodiment of the messaging system of FIG. 9B, wherein the analytics system includes a behavior analytics system and a messaging analytics system.

FIG. 10A is an exemplary block diagram illustrating yet another alternative embodiment of the system of FIG. 9A, wherein the measurement system includes a sensor system, a signal acquisition system and a signal pre-processing system.

FIG. 10B is an exemplary detail block diagram illustrating an alternative embodiment of the measurement system of FIG. 10A, wherein the signal acquisition system further includes analog front-end, signal conditioning, and/or analog-to-digital converter, and/or wherein the pre-processing system includes digital filter and/or signal analysis.

FIG. 11A is an exemplary block diagram illustrating yet another alternative embodiment of the messaging system of FIG. 9A, wherein the behavior analytics system includes a contextual data processing system, and biometric data processing system, a behavior marker extraction system and a behavior modeling system.

FIG. 11B is an exemplary block diagram illustrating an alternative embodiment of the system of FIG. 11A, wherein the behavior analytics system further includes a behavior personalization system.

FIG. 12A is an exemplary block diagram illustrating yet another alternative embodiment of FIG. 9A, wherein the message analytics system includes a behavior interpretation system and a message generator system.

FIG. 12B is an exemplary block diagram illustrating an alternative embodiment of the system of FIG. 12A, wherein the message analytics system further includes a message customization system.

FIG. 12C is an exemplary block diagram illustrating another alternative embodiment of the system of FIG. 12A, wherein the message analytics system further includes a message personalization system.

FIG. 13 is an exemplary detail block diagram illustrating yet another alternative embodiment of the system of FIG. 12A, wherein the message generator system includes message scheduler, message logic and message filter.

FIG. 14 is an exemplary block diagram illustrating yet another alternative embodiment of the messaging system of FIG. 9A, wherein the information system includes a message database, a message history database, an application database, a context database, a user database, a system implementation database and/or a system operation database.

FIG. 15 is an exemplary drawing illustrating a behavior change system according to the messaging method of FIG. 1 and based on the messaging system of FIG. 9A.

FIG. 16A is an exemplary flow diagram illustrating an embodiment of the data flow through the messaging system of FIG. 9A, according to the messaging method of FIG. 1.

FIG. 16B is an exemplary flow diagram illustrating an alternative embodiment of the data flow through the messaging system of FIG. 9A according to the messaging method of FIG. 1, wherein user data, behavior personalization data and message personalization data are used through the data flow.

FIG. 17A is an exemplary drawing illustrating an embodiment of the behavior model generated by the behavior analytics system of FIG. 11A.

FIG. 17B is an exemplary drawing illustrating an alternative embodiment of the behavior model generated by the behavior analytics system of FIG. 11A.

FIG. 18 is an exemplary drawing illustrating an embodiment of the message object format used in the messaging system of FIG. 9A, and according to the messaging method of FIG. 1.

FIG. 19 is an exemplary flow diagram illustrating an embodiment of the messaging analytics of FIG. 13A, wherein the behavior interpretation system consists of a decision tree using the daily values of the behavior markers.

FIG. 20 is an exemplary flow diagram an alternative embodiment of the messaging analytics of FIG. 13A, wherein the behavior interpretation system consists of a decision tree using the last two daily values of the behavior markers.

FIG. 21 is an exemplary flow diagram illustrating another alternative embodiment of the messaging analytics of FIG. 13A, wherein the behavior interpretation system consists of advanced logic.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

Since currently-available messaging systems are incapable of delivering messaging that effectively and sustainably motivate behavior change, a biometric and context based messaging method and system that generates personalized and contextualized messages based on sensor data, can prove desirable for a wide range of applications, such as wellness, fitness, health and healthcare, or to motivate healthy habits or behaviors.

Method

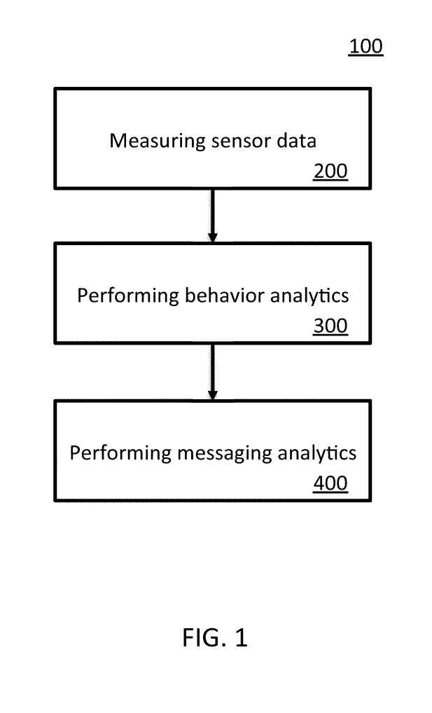

The generic and sophisticated method advantageously generates personalized messages based on biometric and contextual data. This can be achieved, according to one embodiment disclosed herein, by the method 100 for biometric and context based messaging as illustrated in FIG. 1. As shown in FIG. 1, the method 100 can include:

measuring, at 110, sensor data,

performing, at 120, behavior analytics, and

performing, at 130, messaging analytics.

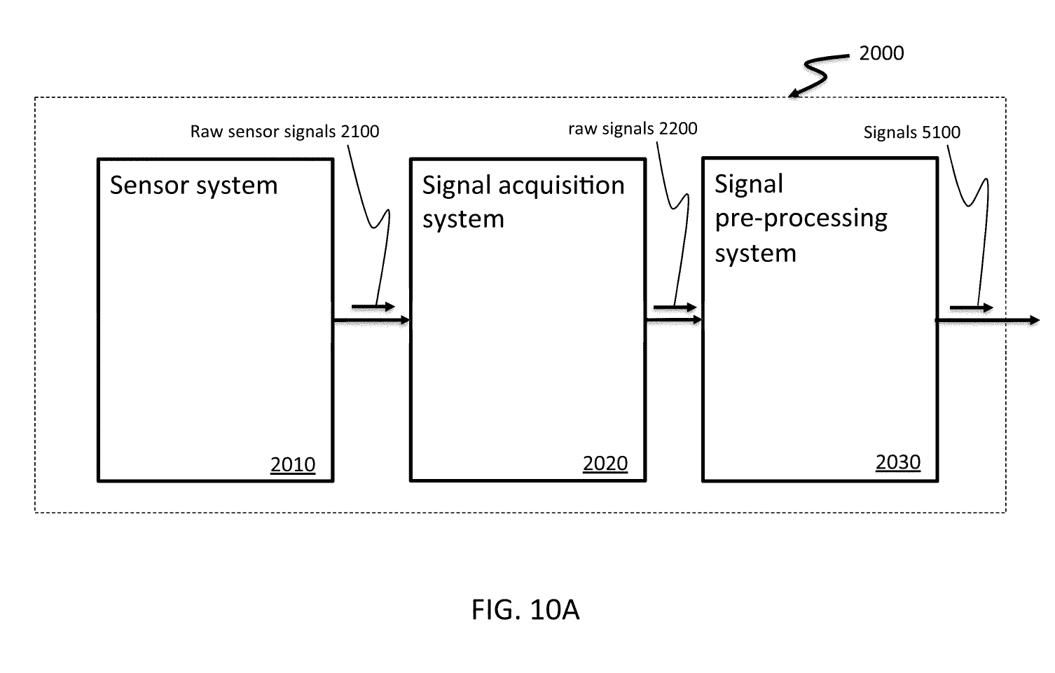

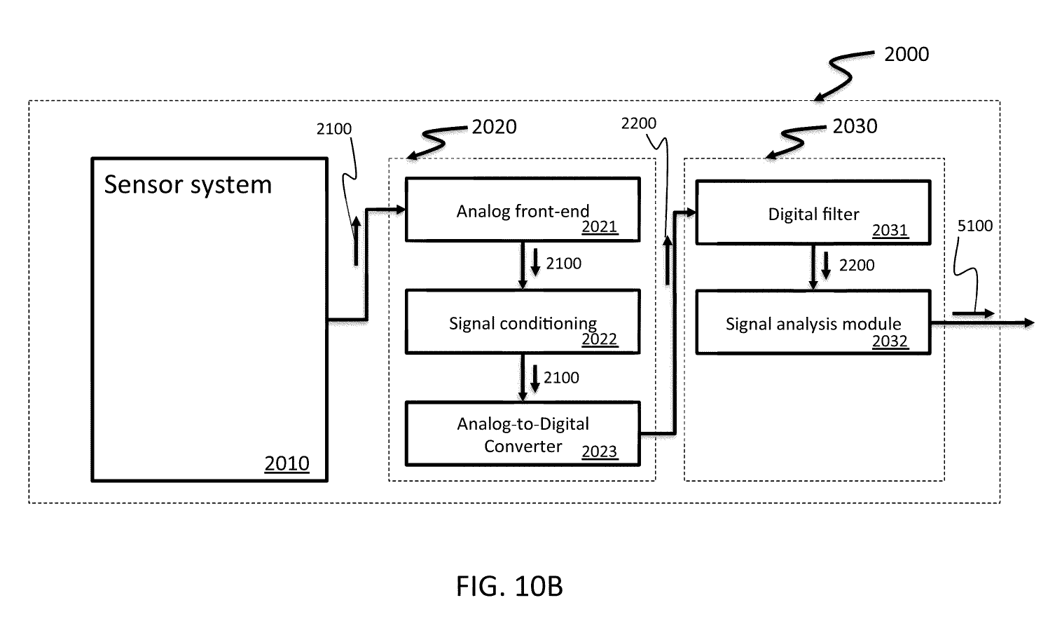

Measuring, at 110, sensor data can be achieved using one or more sensors (not shown). Stated somewhat differently, in one embodiment, the sensor data can be measured, at 110, from a sensor system 2010 (shown in FIG. 10A) that can include one or more sensors. The sensors can include sensors that measure the same and/or different types of sensor data. The method 100 advantageously can be applied to any type of sensors. In one embodiment, sensors embedded in smartphones can be used. For example, an accelerometer or motion sensor embedded in the smartphone can be used to measure movement of a user and/or to measure context. Another example can be to use a Global Positioning System (GPS) device embedded in the smartphone to measure a location of the user. A third example can be to use a camera and a Light Emitting Diode (LED) in the smartphone to measure a pulse rate of the user using a method known as PhotoPlethysmoGraphy (PSG). Using the camera and the LED, and/or other optical sensors embedded in the smartphone, other body parameters can be measured, including, but not limited to, heart rate, heart rate variability, blood flow, blood pressure, blood oxygen levels, blood carbon dioxide levels, blood composition, breathing rate data, breathing depth data, exhaled and inhaled air composition data, skin conductance data, tissue impedance data, electromyogram data, electrocardiogram data, electroencephalogram data, etc. In a further embodiment, ambulatory sensors and/or wearable sensors can be used. For instance, an activity tracker can be used to track the activity of the user, or a pulse monitor can be used to track the heart rate and heart rate variability of the user. Measuring, at 110, sensor data thereby can yield a set of signals that may include but are not limited to, physical signals, physiological signals and/or environment signals, characterizing the user and the environment in which the user evolves.

Performing, at 120, behavior analytics may consist in translating physical, physiological and/or environment signals in a behavior model. Here the term “behavior” should be understood in its broadest sense, including single or multiple parameters and/or including static and dynamic views on at least one of health, wellness, lifestyle and healthcare of the user. Behavior may, for instance, be the level of activity of the user over a predetermined time period such as an hour, a day, a week a month, or even shorter or longer time periods. Behavior may also be the evolution of the user's activity over time. In another example, behavior may include multiple lifestyle parameters such as activity, sleep and/or stress, taken as single measurements in time or as trends over time. Behavior may also be a set of the user's vital signals, at a given point in time or as trends over time. Behavior may also be the status of a chronic disease, at a given point in time, or as a trend over time. Behavior may include environment factors such as, for instance, location, time, weather, pollution levels, etc. Performing, at 120, behavior analytics may take one or more physical, physiological and/or environment signals as input, and yield a behavior model at the output.

Performing, at 130, messaging analytics may consist in translating a behavior model in one or more messages. The messages preferably are personalized because the messages relate to the user's behavior captured when performing, at 120, behavior analytics.

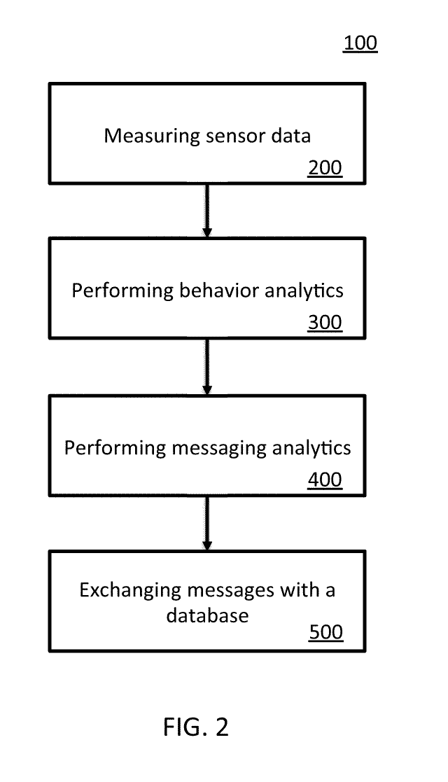

FIG. 2 shows an alternative embodiment of the method 100 for biometric and context based messaging of FIG. 1. As illustrated in FIG. 2, the method 100 can include exchanging messages, at 140, with a database. The database can be a message database in which messages are stored, and exchanging messages, at 140, can include getting a message from the database. The database can be a message history database in which a history of the messages delivered to the user is stored, and exchanging messages, at 140, can include writing a message to the message history database.

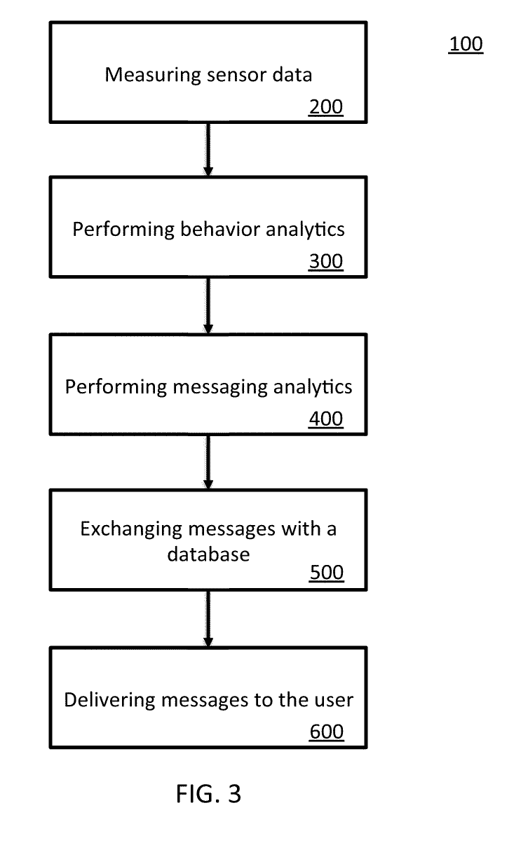

FIG. 3 shows an alternative embodiment of the method 100 for biometric and context based messaging of FIG. 2. As illustrated in FIG. 3, the method 100 can include delivering messages to the user, at 150. Delivering messages to the user, at 150, can be done in any suitable format and on any conventional platform. For instance, the messages can be delivered as notifications, text messages, voice messages, multimedia messages, and/or emails, delivered on a cellular phone, smartphone, tablets, computer, smart-watch and/or smart-glasses.

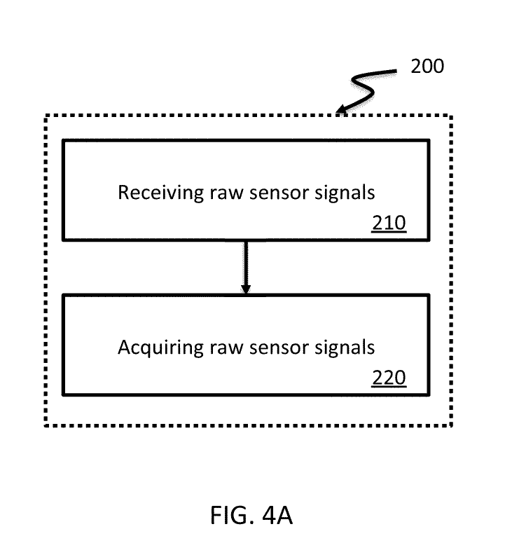

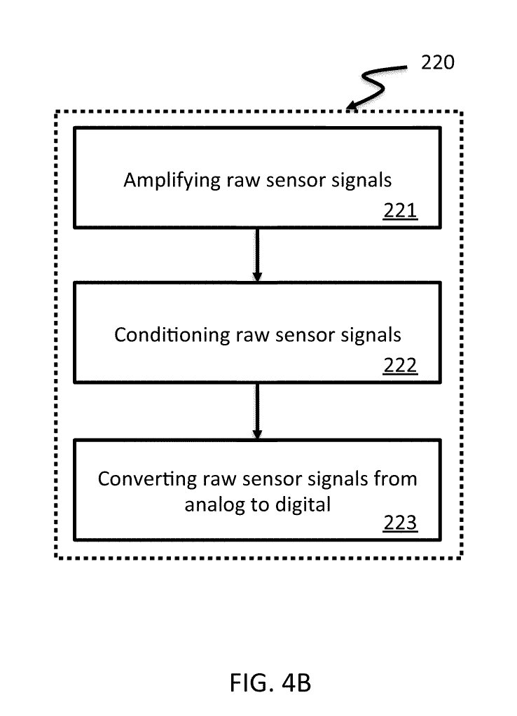

FIG. 4A shows another alternative embodiment of the method 100 for biometric and context based messaging of FIG. 1, wherein measuring, at 200, sensor data, can include:

receiving, at 210, raw sensor signals, and

acquiring, at 220, raw sensor signals from the one or more sensors.

FIG. 4B shows an alternative embodiment of the method 100 for biometric and context based messaging of FIG. 4A, wherein acquiring, at 220, raw sensor signals can further include, when dealing with analog sensors,

amplifying, at 221, the raw sensor signals,

conditioning, at 222, the raw sensor signals, and

converting, at 223, the raw signals from analog to digital domain.

Conditioning, at 222, the raw sensor signals can include filtering, scaling, chopping and any other conventional techniques used to measure sensitive analog signals.

Receiving, at 210, raw sensor signals can involve receiving raw sensor signals from one or more sensors. Receiving, at 210, raw sensor signals can include receiving analog raw sensor signals and/or digital raw sensor signals, and can include one or multiple raw sensor signals, and one or multiple channels in each signals.

Acquiring, at 220, raw sensor signals can include reading the raw sensor signals received at 210, and amplifying and/or preparing the raw sensor signals for future analysis and processing.

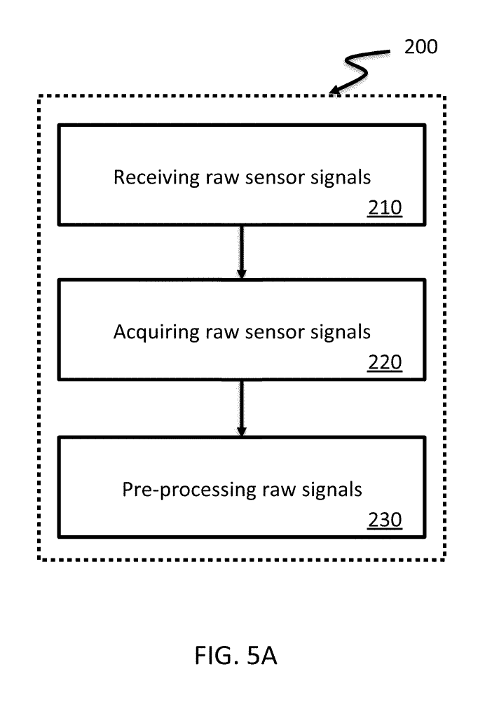

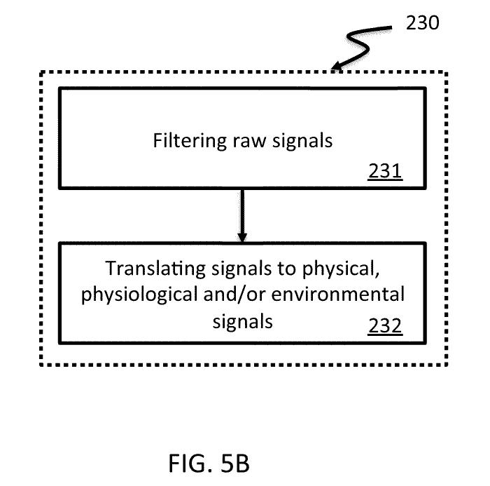

As shown in FIG. 5A, measuring sensor data, at 200, may also include pre-processing, at 230, raw signals, to yield a set of signals that may include, but are not limited to, physical, physiological and/or environmental signals.

As further detailed and shown in FIG. 5B, in an alternative embodiment of the method 100 for biometric and context based messaging of FIG. 5A, pre-processing, at 230, raw signals can include:

filtering, at 231, raw signals, and

translating, at 232, signals to physical, physiological and/or environmental signals.

Filtering, at 231, raw signals can be applied in the frequency domain, in the time domain, or in the time-frequency domain. Filtering can be used to reduce measurement artifact, reduce measurement noise, separate multiple signals from the same sensors, and/or to improve the performances of the system in any aspects.

Translating, at 232, raw signals to physical, physiological and/or environmental signals can be achieved by selecting sensor signals, or combining multiple sensor signals to represent physical, physiological and/or environmental signals.

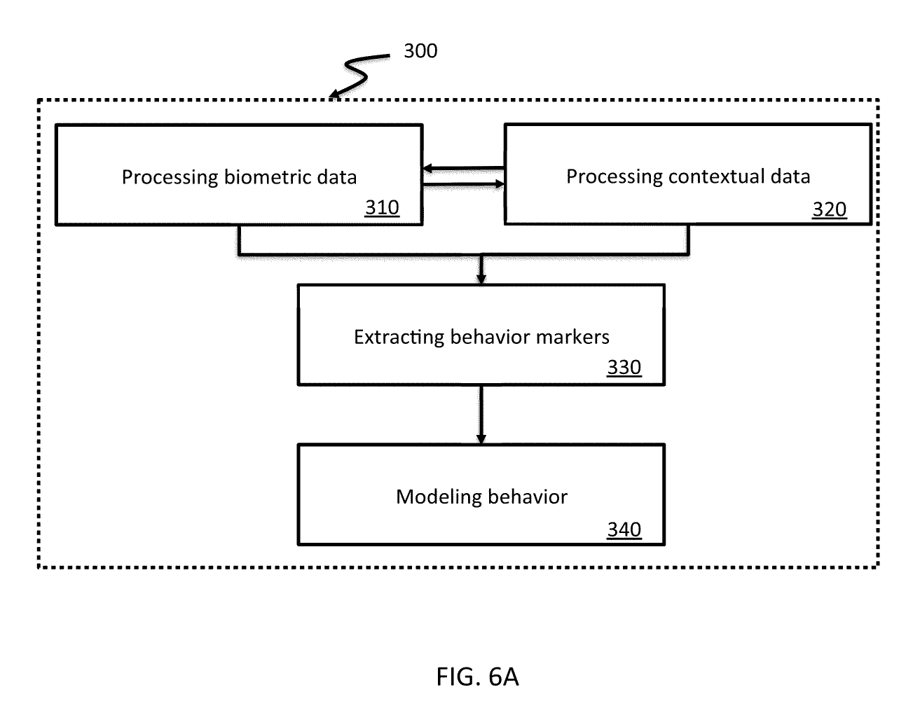

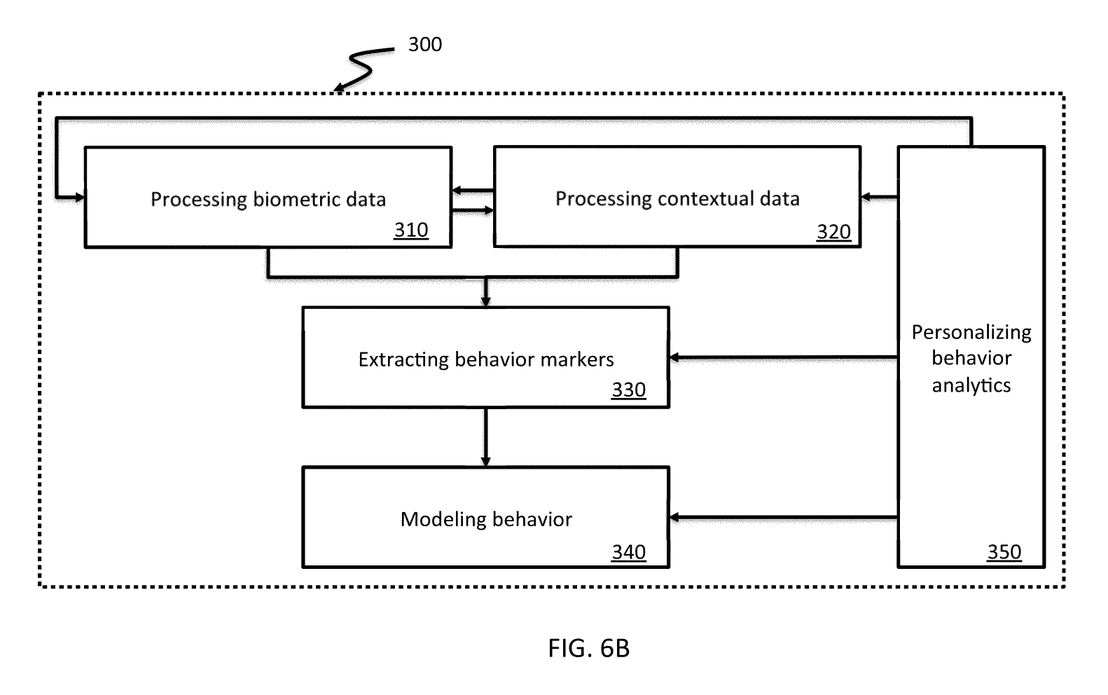

FIG. 6A shows yet another alternative embodiment of the method 100 for biometric and context based messaging of FIG. 1, wherein performing, at 300, behavior analytics can further comprise at least one of:

Processing, at 310, biometric data, wherein the pre-processed physical, physiological and environment signals are converted to biometric data,

Processing, at 320, contextual data, wherein pre-processed physical, physiological and environment signals are converted to contextual data,

Extracting, at 330, behavior markers, wherein biometric data and contextual data are converted to behavior markers, and

Modeling, at 340, behavior, wherein behavior markers are converted to behavior models.

Processing, at 310, biometric data can extract biometric data from the physical, physiological and/or environmental signals. Biometric data can include, but are not limited to, activity counts, steps, movements, activity types, activity intensity, energy expenditure, calorie burned, heart rate, heart rate variability, systolic and diastolic blood pressure levels, and/or blood glucose levels. Biometric data can be computed using time-domain, frequency-domain or time-frequency processes. The manner by which the biometric data is processed, at 310, can adapt based on contextual data or based on the user data.

Processing, at 320, contextual data can extract contextual data from the physical, physiological and/or environmental signals. Contextual data can include, but is not limited to, activity of the user, daily routines of the user, location, time, and/or social media activity. Contextual data can be computed using time-domain, frequency-domain or time-frequency processing algorithms. The manner by which the contextual data is processed, at 320, can adapt based on processing biometric data or based on user data.

Extracting, at 330, behavior markers can take the biometric and contextual data, and further process, analyze and/or combine them into behavior markers. The behavior markers can be seen as contextualized version of the biometric data, i.e. biometric data that has been corrected for the context. Behavior markers can also include combination of individual markers. Furthermore, behavior markers can also include patterns of behavior markers and/or combinations of behavior markers over time.

Modeling, at 340, behavior advantageously can use behavior markers to compute a model of the user's behavior. A behavior model can be a matrix representation of behavior markers at a given point in time and/or a matrix representation of behavior markers over time. Modeling, at 340, behavior can use algorithms from the machine learning field to generate behavior models, including but not limited to, decision trees, support vector machines, Markov chains, Bayesian probabilistic models, hierarchical models, etc.

FIG. 6B shows a further embodiment of performing, at 300, behavior analytics, that can also comprise personalizing, at 350, behavior analytics. Personalizing, at 350, behavior analytics can use the user data and/or contextual data to personalize the manner in which the biometric data is processed, at 310, the manner in which the contextual data is processed, at 320, the manner in which the behavior markers are extracted, at 330, and/or the manner in which the behaviors are modeled, at 340. For example, when applying the method 100 for biometric and context based messaging to an athlete during a training program, his weight, BMI and fitness levels will change over time, and processing, at 310, biometric data can take these changes into account to generate more accurate data. In this case, personalizing, at 350, behavior analytics can adapt processing, at 310, biometric data based on changes in the user data. In another example, the behavior markers can change depending on the context. For example, heart rate variability is a good marker for stress in rest conditions but is influenced by physical activity during exercise. In this case, personalizing, at 350, behavior analytics can adjust extracting, at 330, behavior markers based on contextual data. Personalizing, at 350, behavior analytics can use context and user data to adapt modeling behavior, at 360. For instance, specific biometric data may be more or less relevant to the user's behavior depending on the context.

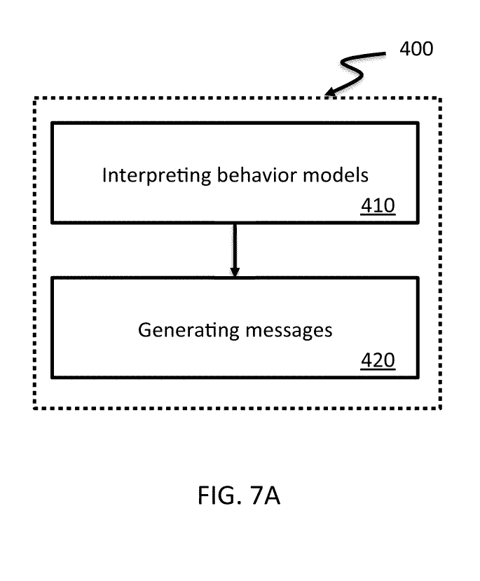

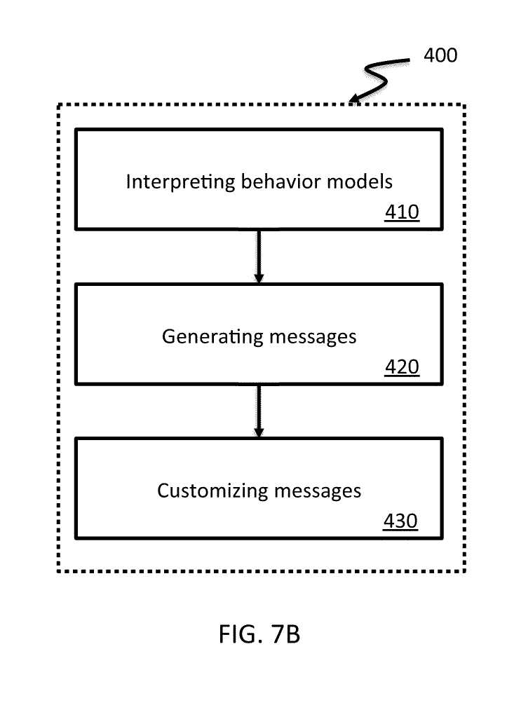

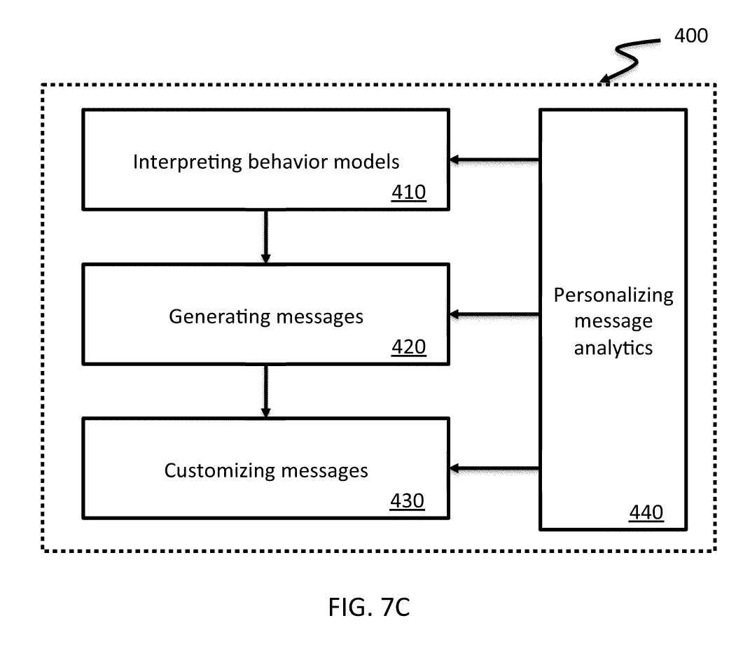

FIG. 7A shows yet another alternative embodiment of the method 100 for biometric and context based messaging of FIG. 1, wherein performing, at 400, messaging analytics can further include:

Interpreting, at 410, behavior models, and

Generating, at 420, messages.

Interpreting, at 410, behavior models can be achieved by converting behavior models to message attributes, where message attributes can include a set of words and labels that describe the characteristics of the messages that should be delivered to the user given her behavior as modeled using the messaging method 100. For instance, message attributes may qualify the level of activity, sleep, blood pressure, blood glucose, and/or stress of the user with words such as “increased”, “decreased”, “stable.” In another example, message attributes may specify the time at which a message should be delivered. Generating, at 420, messages can be achieved by converting the message attributes in an actual message. The message can be accompanied with its metadata. The message can include the actual text of the message. The message metadata can include information about the message.

FIG. 7B shows an alternative embodiment of performing, at 400, messaging analytics that can include customizing, at 430, messages. Customizing, at 430, messages can be achieved by adapting a message to the user's preferences or to the user's specific data. For instance a user may prefer a specific tone for the message, such as authoritative, friendly, imperative, etc.

FIG. 7C shows another alternative embodiment of performing, at 400, messaging analytics that can include personalizing, at 440, message analytics, wherein interpreting, at 410, behavior models, generating, at 420, messages, and/or customizing, at 430, messages can be personalized using the user data and/or the contextual data. For instance, interpreting, at 410, behavior models can be done differently depending of the weight or body mass index of the user. For example, a slight increase in activity levels may, for a user with a high body mass index, be interpreted as a significantly positive improvement in behavior. Whereas the same increase in activity for a person with a normal body mass index may not be as significant. Similarly, generating, at 420, messages can be done differently depending on the body mass index, even when the measured behavior model is similar. For instance, when a decreased activity level is observed, the obese user may receive messages adapted to overweight users, and such messages may be different than the ones for healthy users.

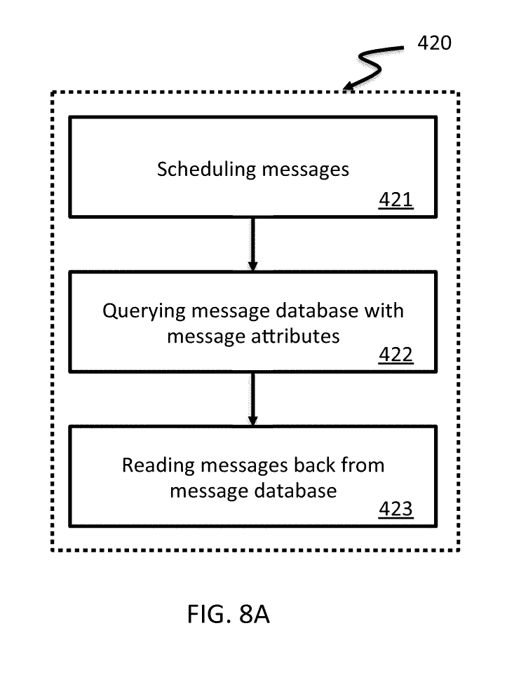

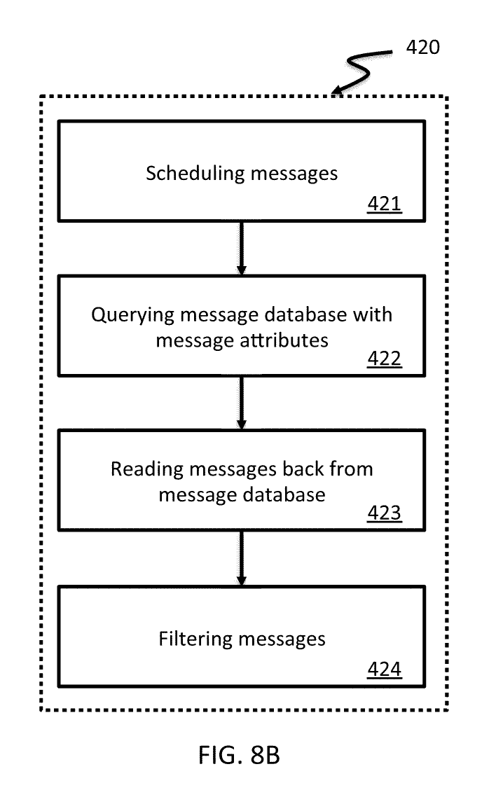

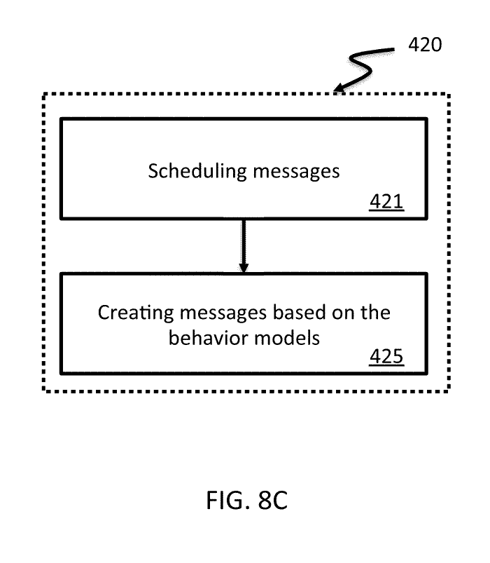

FIG. 8A shows an alternative embodiment of generating, at 420, messages that can further include:

scheduling, at 421, messages,

querying, at 422, message database with message attributes, and

reading, at 423, messages back from the message database.

Scheduling, at 421, messages can define when to generate a message. Messages can be scheduled for delivery at specific predefined times of the day. Or messages can be scheduled in response to specific user actions, for example, when the user checks her smartphone or when she is opening a mobile application. Alternatively and advantageously, messages can be scheduled according to the user's behavior models and/or according to a specific context, such as the user's daily routines or the user's location. Querying, at 422, message database with message attributes can consist in comparing message attributes with the metadata associated with each message, and returning messages for which the metadata matches the message attributes. Reading, at 423, messages back from the message database can consist in receiving and caching the messages returned by querying, at 422, message database. The read messages can then be available for further processing and/or delivering to the user.

FIG. 8B shows another alternative embodiment of generating, at 420, messages that can include filtering, at 424, messages. Messages may be filtered, at 424, based on messaging history, user's data and/or user feedback, to select one or more messages to be delivered to the user. For example, filtering messages may be achieved by further specifying message attributes based on messaging history, user's data and/or user feedback, and selecting the messages for which the metadata matches the further specified message attributes.

FIG. 8C shows yet another alternative embodiment of generating, at 420, messages that can include:

scheduling, at 421, messages, and

creating, at 425, messages based on the behavior models.

Creating, at 425, messages based on the behavior models can be done, for instance, using dynamic creation of messages based on the message attributes, or using semantic inference to automatically create a message related to the attributes.

System

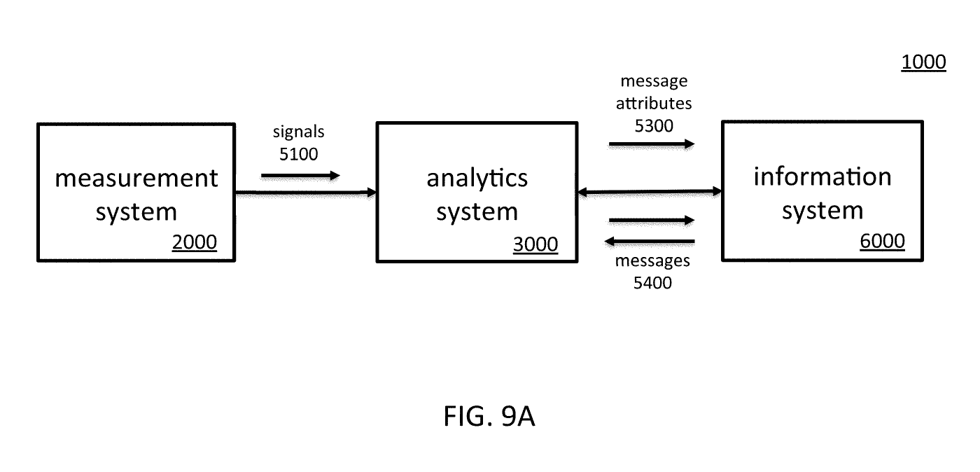

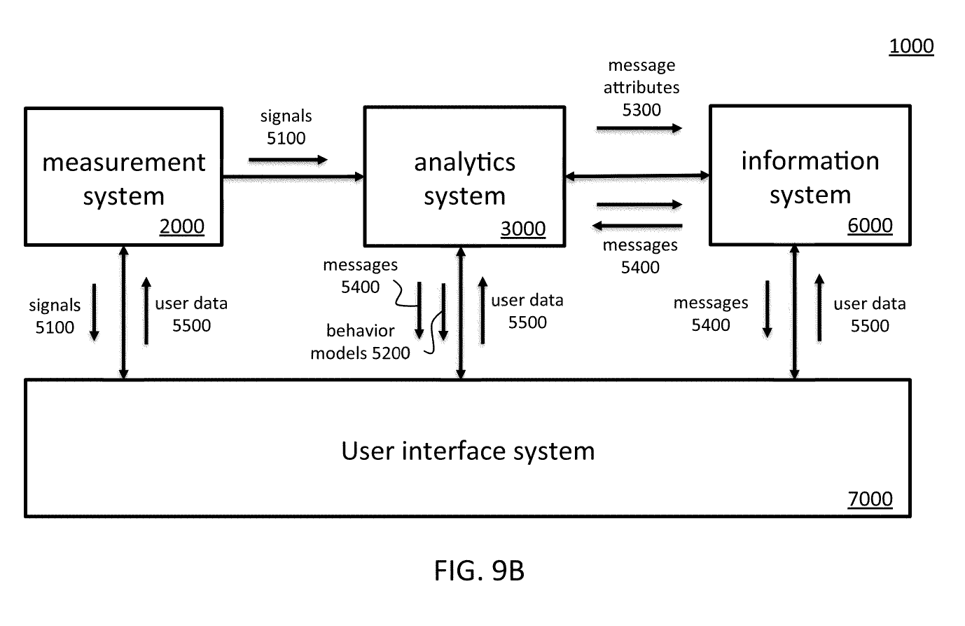

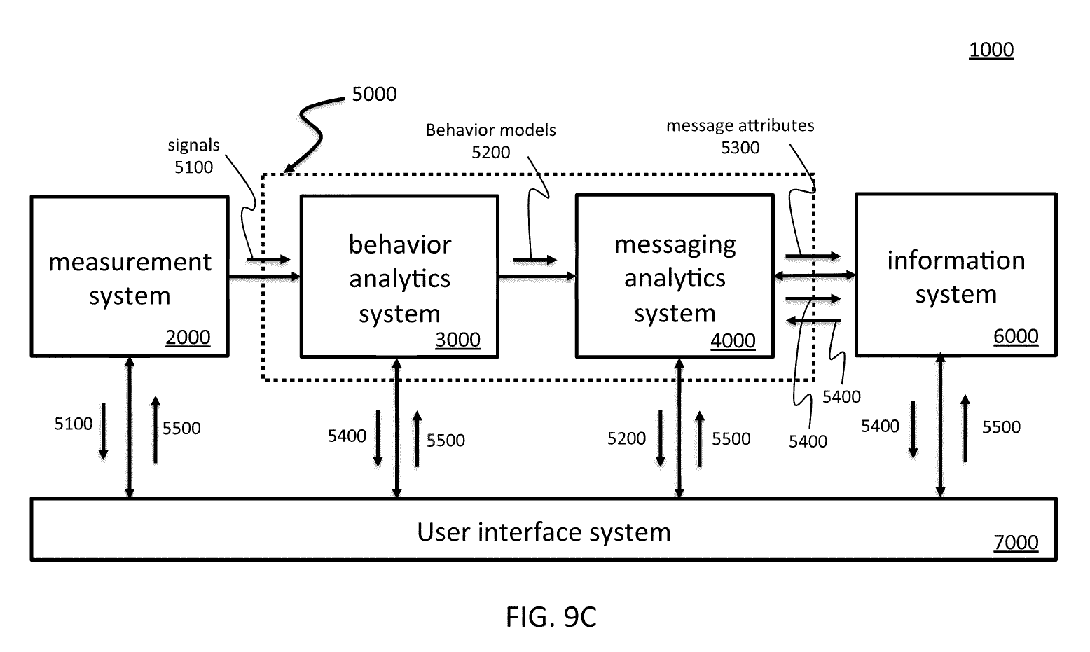

The method 100 described above for biometric and context based messaging can be achieved, according to one embodiment disclosed herein, by a messaging system 1000. One exemplary embodiment of the messaging system 1000 is illustrated in FIG. 9.

Turning to FIG. 9, the messaging system 1000 is shown as including at least a measurement system 2000, an analytics system 5000 and an information system 6000. The measurement system 2000 can include one or more sensors and can measure and pre-process signals from the sensors. The outputs of the measurement system 2000 are signals 5100, which can be any physical, physiological and/or environmental signals. The analytics system 5000 analyses the signals 5100, and extracts information about wellness, fitness, health or healthcare of the user. Additionally, and/or alternatively, the information extracted from the signals 5100 can include information about behavior, lifestyle and habits of the user. The analytics system 5000 thereby can define message attributes 5300. The message attributes 5300 are used by the information system 6000 to generate messages 5400. The messages 5400 may then be passed back to the analytics system 5000. In one embodiment, still illustrated on FIG. 9, the analytics system 5000 generates the messages 5400 and passes them to the information system 6000. The information system 6000 can also store the messages 5400, information about the messages 5400, as well as general and/or field specific information related to behavior and/or behavior change. FIG. 9 illustrates an exemplary manner by which the messaging system 1000 creates and generates messages 5400 based on the sensor data hence realizing a goal of biometric and context based messaging.

FIG. 9B shows an alternative embodiment of the messaging system 1000, in which the messaging system 1000 includes a user interface system 7000 for interacting with the user. The user interaction can be achieved at one or multiple levels. The user interface system 7000 can be used to deliver the messages 5400 to the user. Alternatively or additionally, the user interface system 7000 can be used to report the behavior models 5200 to the user. Alternatively or additionally, the user interface system 7000 can be used to visualize the signals 5100 for the user. Alternatively or additionally, the user interface system 7000 may be used to report or display to the user any information about the messaging system 1000. In a further embodiment, the user interface system 7000 can collect user data 5500. The user data 5500 may include, but is not limited to, anthropomorphic data, information about the user's habits, lifestyles or daily routines, information about the user's specific medical conditions, information about the user's environment, etc. Alternatively or additionally, the user data 5500 can include user feedback. The user feedback can be collected under the format of questions (prompted) or as open text (spontaneous). Questions can be generated by the analytics system 5000. Questions can be predefined, can change over time based on a predefined schedule, can adapt based on a message 5400 or a series of messages 5400 that are delivered to the user, or can adapt based on behavior as analyzed by the analytics system 5000. The user feedback can include feedback on the messages 5400 that are being delivered. Alternatively or additionally, the user feedback can include information on the user's behavior, experience and/or feelings at a particular moment in time, or in the past. For example, users can be asked how they are feeling at the moment they read the message, or if they are feeling stress right now, or if they have experienced stressed in the last hour or in the course of the day. In another example, users can be asked if they have taken a specific food item today, or if they have smoked today. In another example, users can be asked to enter information about what they have eaten today, or about specific activities they have performed. The user data 5500 can be used as input to the analytics system 5000 in addition to the signals 5100. The user data 5500 can also be used by the measurement system 2000 and/or by the information system 6000.

FIG. 9C shows another alternative embodiment of the messaging system 1000, in which the analytics system 5000 is split into two systems: a behavior analytics system 3000 and a messaging analytics system 4000. The behavior analytics system 3000 can process, analyze and/or interpret the signals 5100 coming from the measurement system 2000, to create the behavior models 5200 for the user. The behavior analytics system 3000 can also personalize and adapt the behavior models 5200 to the user. The message analytics system 4000 can create message attributes 5300 and/or messages 5400 based on the behavior models 5200. The message analytics system 4000 can also personalize and adapt the messages 5400 to the user, and decide when and how to deliver the messages 5400 as to maximize messaging efficacy.

FIG. 10A is a detailed drawing showing yet another alternative embodiment of the messaging system 1000 of FIG. 9A. As illustrated in FIG. 10A, the measurement system 2000 can include a sensor system 2010, a signal acquisition system 2020 and a signal pre-processing system 2030. The sensor system 2020 can include one or more sensors. The term “sensor” comprises a conventional type of sensor for measuring, including but not limited to sensors producing waveforms representing biological, physiological, neurological, psychological, physical, chemical, electrical and mechanical signals, such as pressure, sound, temperature and the like, probes, surveillance equipment, measuring equipment, and any other means for monitoring parameters representative of or characteristic for an application domain. When applied to the field of wellness, lifestyle, health or healthcare, for example, the sensors can typically be used to measure body signals and the surrounding environment. The sensors can sense physical, physiological and/or environmental signals. In one embodiment, the one or more sensors may be embedded in a smartphone, be carried by the user (ambulatory sensors) or be worn by the user (wearable sensors). Smart phone sensors can include, but are not limited to: accelerometers, cameras, Light Emitting Diodes (LEDs), and/or microphones. For the purpose of this disclosure, Global Positioning System (GPS), Bluetooth and other wireless communication devices can also be considered as sensors. Ambulatory sensors can include, but are not limited to, blood pressure monitors, blood glucose monitors, biochemical assays to measure specific hormones such as cortisol, melatonin, etc. Wearable sensors can include, but are not limited to, activity trackers, heart rate trackers (chest strap or wrist based), ECG monitors, EEG monitors, etc. The sensor system 2010 yields raw sensor signals 2100 as its output.

The signal acquisition system 2020 can interface with the sensor system 2010 and takes the raw sensor signals 2100 as input to convert them into raw signals 2200.

The signal pre-processing system 2030 can take the raw signals 2200 as input, and pre-process them to output the signals 5100. The signals 5100 can be of any nature, for instance, physical, physiological and/or environmental signals.

FIG. 10B is a detailed drawing illustrating an alternative embodiment of the measurement system 2000 of FIG. 10A. As illustrated on FIG. 10B, the signal acquisition system 2020 can include an analog front-end 2021, signal conditioning 2022 and an Analog to Digital Converter (ADC) 2023. In another alternative embodiment, again illustrated on FIG. 10B, the pre-processing system 2030 can include a digital filter 2031 and a signal analysis module 2032. The signal analysis module 2032 can combine and/or convert the raw signals 2200 into the physical, physiological and environmental pre-processed signals 5100.

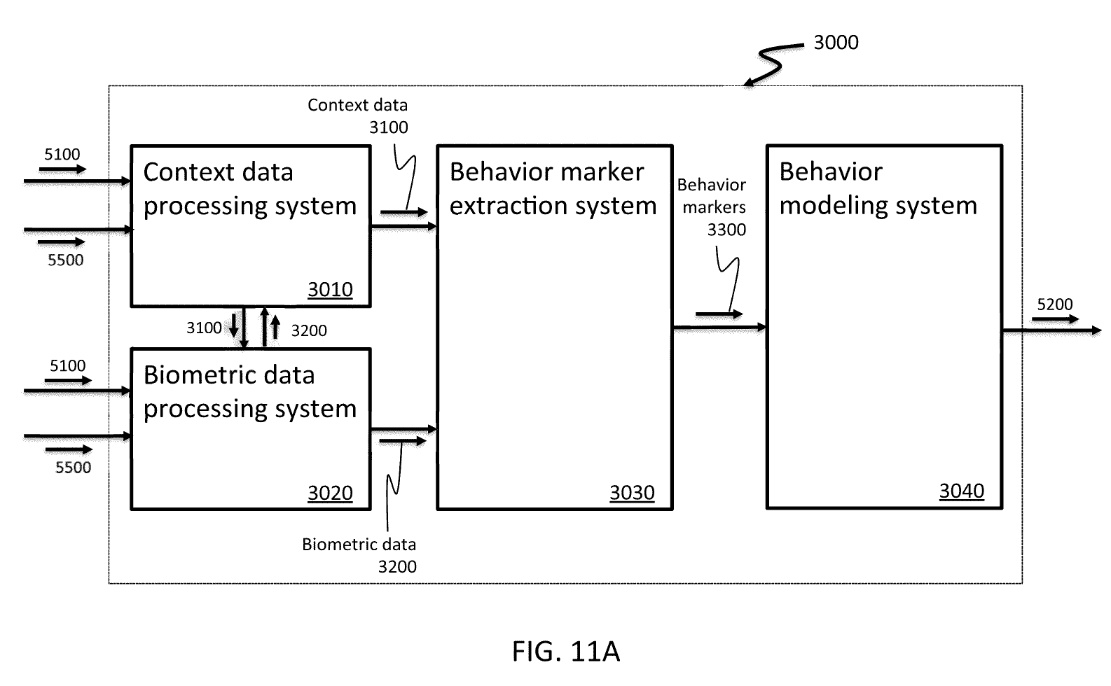

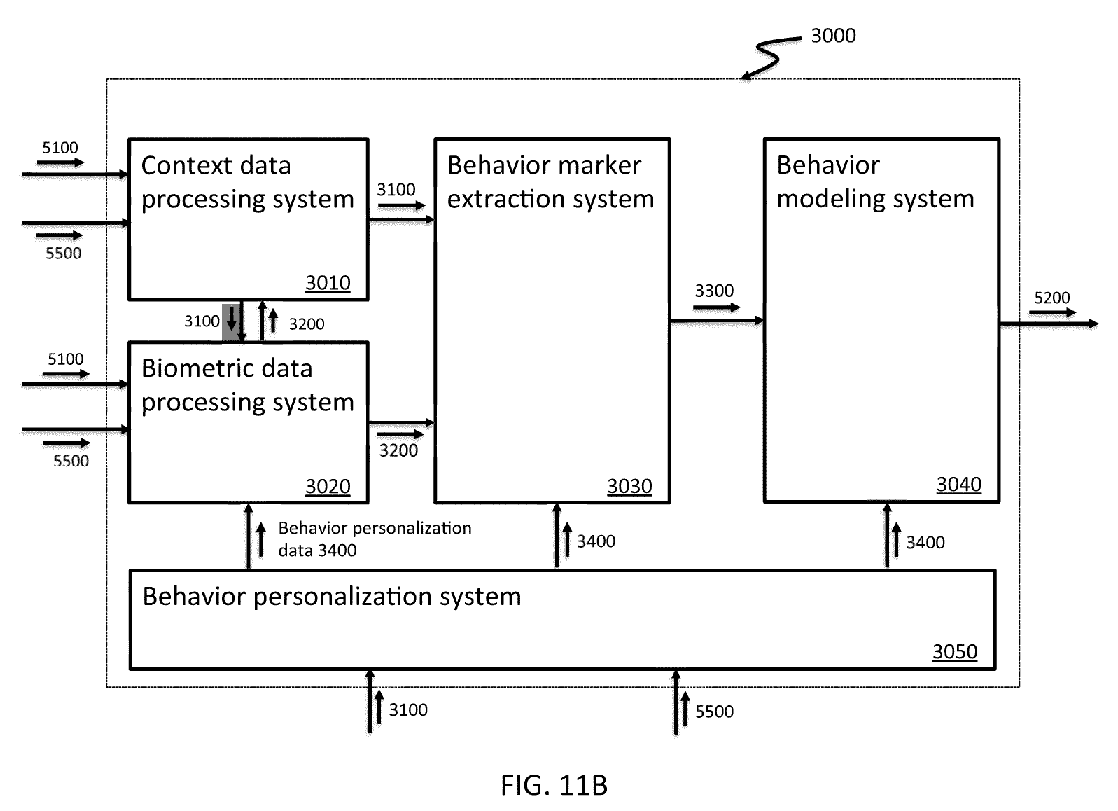

FIG. 11A is a detailed drawing showing yet another alternative embodiment of the messaging system 1000 of FIG. 9A. As illustrated on FIG. 11A, the behavior analytics system 3000 can include a contextual data processing system 3010, a biometric data processing system 3020, a behavior marker extraction system 3030 and a behavior modeling system 3040.

The contextual data processing system 3010 can take the physical, physiological and environmental pre-processed signals 5100 as input, and process the signals 5100 to yield the contextual data 3100 as the output. The contextual data processing system 3010 can also take the user data 5500 as input. The user data 5500, for example, can include anthropomorphic data, or information about the context that has been manually entered by the user. Examples of contextual data 3100 may include, but are not limited to: daily routines, location trajectories or maps, noise data, light data, pollution data, weather maps, etc.

The biometric data processing system 3020 can take the physical, physiological and environmental pre-processed signals 5100 as input, and process them to yield the biometric data 3200 as the output. The biometric data processing system 3020 can also take user data 5500 as input. The user data 5500, for example, can include anthropomorphic data, or biometric information that has been manually entered by the user. Examples of biometric data 3200 may include, but are not limited to, activity profile (amount of activity over time), heart rate, heart rate variability, muscle activity, energy expenditure over time, calories burnt over time, galvanic skin response, brain activity, blood pressure profile, blood glucose profile, etc.

As illustrated on FIG. 11A, the biometric data processing system 3020 can also take the contextual data 3100 as input. The contextual data 3100 can be used to improve the accuracy of the biometric data processing system 3020, by adding contextual information around physical or physiological signals that may affect how physical or physiological signals relate to biometric information. For example, to compute a specific biometric data point 3200 such as a heart rate at rest, it may be useful to know from the contextual data 3100 when the user is at rest, such that those moments are kept for estimating heart rate at rest; whereas, other moments are discarded. Similarly, the contextual data processing system 3010 can take the biometric data 3200 as input. For example, the biometric data 3200 can be used to provide additional information relevant to the context such as level of activity or daily routines.

The behavior marker extraction system 3030 takes contextual data 3100 and biometric data 3200 as inputs, and generates one or more behavior markers 3300. In the area of wellness, fitness and health, behavior markers 3300 can be physical, physiological, psychological, psycho-physiological, mental, biological, contextual and/or environment markers of the user's behaviors. Practical examples may include, but are not limited to, number of steps, time active, time spent doing specific physical activities or exercises, calories burnt over a defined period of time, sleep time, sleep quality, time in deep sleep, time in light sleep, cardio-respiratory fitness, cardio-vascular fitness, VO2 max, stress level, relaxation level, weight gain, dietary intake, hypertensive state, hypoglycemic state, hyperglycemic state, etc.

The behavior modeling system 3040 can take the behavior markers 3300 as input, and combine them into one or more behavior models 5200. The behavior models 5200 can capture the user's behavior over a certain period of time and until now. In one example, the behavior model 5200 can capture the user's behavior over the last day. In another example, the behavior model 5200 may capture the user's behavior from the time the user started using the system until now.

FIG. 11B is a detailed drawing showing an alternative embodiment of the behavior analytics system 3000 of FIG. 11A, wherein the behavior analytics system 3000 can include a behavior personalization system 3050. The behavior personalization system 3050 can use the user data 5500 and/or the contextual data 3100 to personalize the biometric data processing system 3020, the contextual data processing system 3010, the behavior marker extraction system 3030 and/or the behavior modeling system 3040. The manner in which the behavior personalization system 3050 impacts the biometric data processing system 3020, the contextual data processing system 3010, the behavior marker extraction system 3030 or the behavior modeling system 3040 can be through the intermediary of the behavior personalization data 3400 that is created by the behavior personalization system 3050 based on the user data 5500.

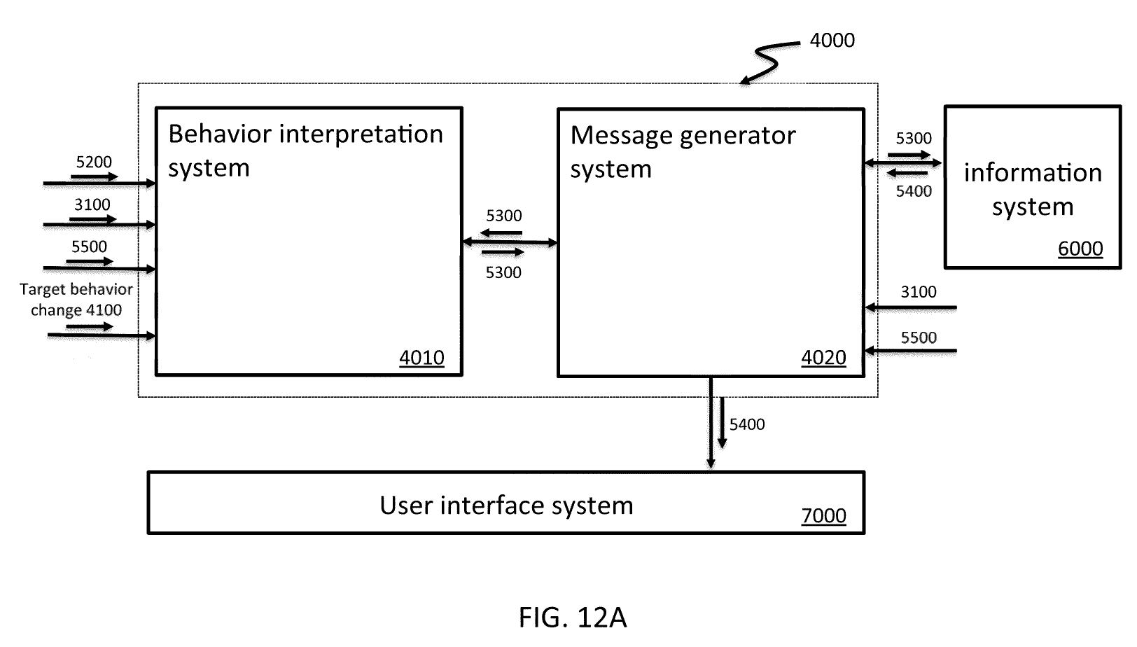

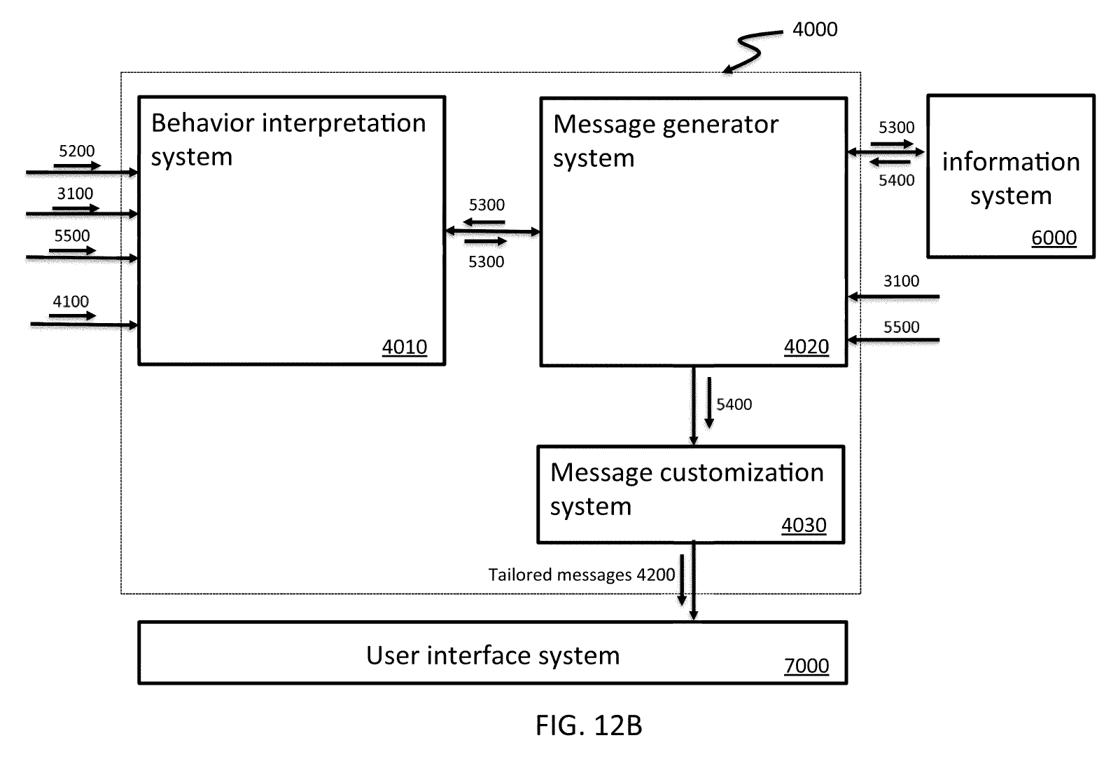

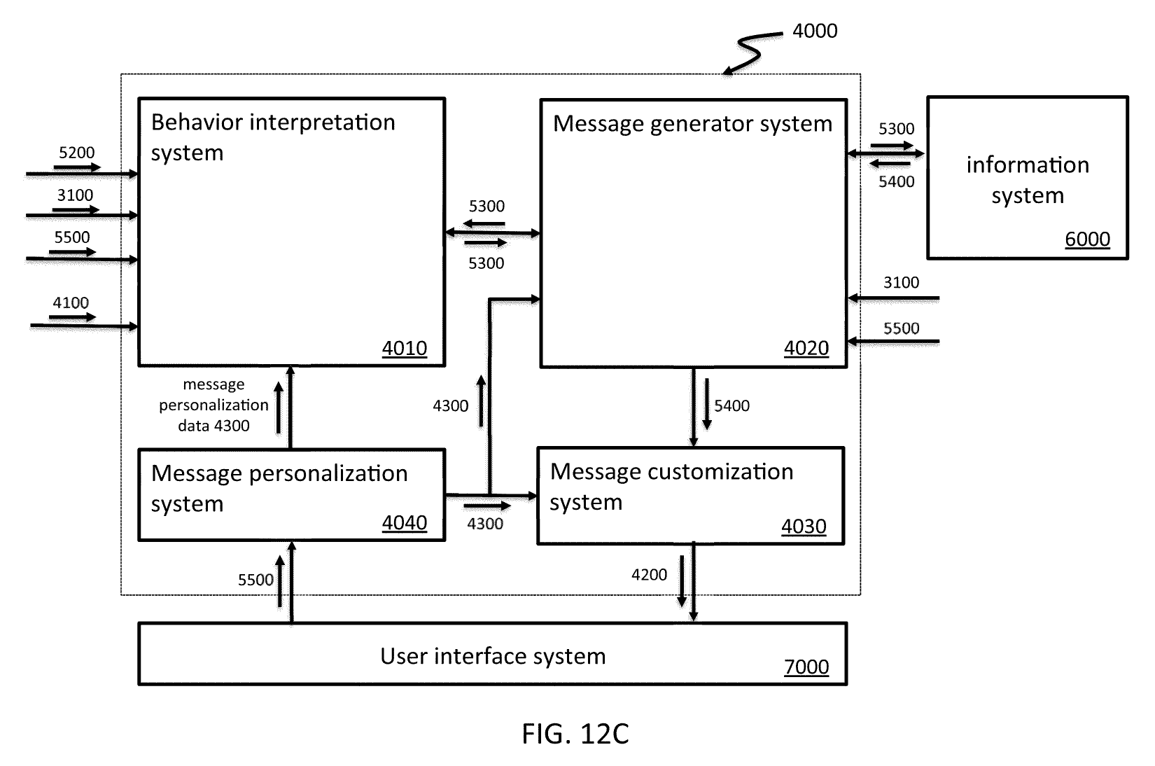

FIG. 12A is a detailed drawing showing yet another alternative embodiment of the messaging system 1000 of FIG. 9A. As illustrated on FIG. 12A, the messaging analytics system 4000 can include a behavior interpretation system 4010 and a message generator system 4020.

The behavior interpretation system 4010 can analyze the behavior models 5200 generated by the behavior analytics system 3000, and define message attributes (MAs) 5300. The MAs 5300 are specific attributes that qualify and/or describe specific characteristics of the messages 5400 that can be delivered to the user. In addition to the behavior models 5200, the behavior interpretation system 4010 can receive the following inputs: contextual data 3100, user data 5500, and target behavior change 4100. The user data 5500 may contain information about the user such as anthropomorphic data, specific medical or health conditions, or any peculiarities related to the users and that may be relevant to analyzing her behavior. The target behavior change 4100 can be defined by the target application, e.g. stop smoking, get a more balanced lifestyle, lose weight, etc. The behavioral interpretation system 4010 can analyze the behavior models 5200 and detect trends in the user's behavior that may be related to changes in the user's physiology, in the environment and/or in the user's behavior change interventions. These trends are used to define the MAs 5300. The behavior interpretation system 4010 can also analyze the evolution of the behavior over time and in relation to messages 5400 that may have been delivered previously, enabling a better understanding of the efficacy of specific messages 5400 on the user that may be taken into account when defining the MAs 5300.

The message generator system 4020 can define and/or use the MAs 5300 to generate the messages 5400 to be delivered to the user. The message generator system 4020 can interact with the behavior interpretation system 4010 to assign values to specific fields of the MAs 5300. The message generator system 4020 can interact with the information system 6000 to access messages 5400 or other content information.

FIG. 12B is a detailed drawing illustrating an alternative embodiment of the messaging analytics system 4000 of FIG. 12B. As illustrated on FIG. 12B, the messaging analytics system 4000 can also comprise a message customization system 4030. The message customization system 4030 can receive the message 5400 from the message generator system 4020 and customize the received message 5400 to provide a tailored message 4200 based on user feedback. The customization of the received message 5400 can be performed at the level of semantics, tone and/or format of the received message 5400.

FIG. 12C is a detailed drawing illustrating another alternative embodiment of the messaging analytics system 4000 of FIG. 12A. As illustrated on FIG. 12C, the messaging analytics system 4000 can also comprise a message personalization system 4040. The message personalization system 4040 advantageously brings a dimension of adaptability in the messaging system 1000. The message personalization system 4040 can take the user data 5500, and preferably user feedback, into account and personalize the messaging analytics system 4000 accordingly. The message personalization system 4040 can affect one or more elements of the message analytics system 4000. The user data 5500 can include user feedback on multiple aspects of the system such as the accuracy of the behavior analytics system 3000 (i.e., how accurate were the latest behavior models 5300 in comparison to what the user actually experienced), content, tone, time of the message, the level of involvement in behavior change, overall feeling about the system, etc. For example, the message personalization system 4040 can control parameters of the behavior interpretation system 4010, for instance by adjusting the interpretation based on the level of correctness of the model as reported by the user through the user interface system 7000. In another example, the message personalization system 4040 can control parameters of the message generator system 4020, for instance by modifying message meta-data based on the user data 5500. In another example, the message personalization element 4040 can control the parameters of the message generator element 4020, for instance by adapting the time of message delivery based on user's preferences. In another example, the message personalization system 4040 can control parameters of the message customization system 4030, for instance by adapting the tone of the message based on user's preferences as contained in the user data 5500. The manner in which the message personalization system 4040 impacts the behavior interpretation system 4010, the message generator system 4020 or the message customization system 4030 can be through the intermediary of the message personalization data 4300 that is created by the message personalization system 4040 based on the user data 5500.

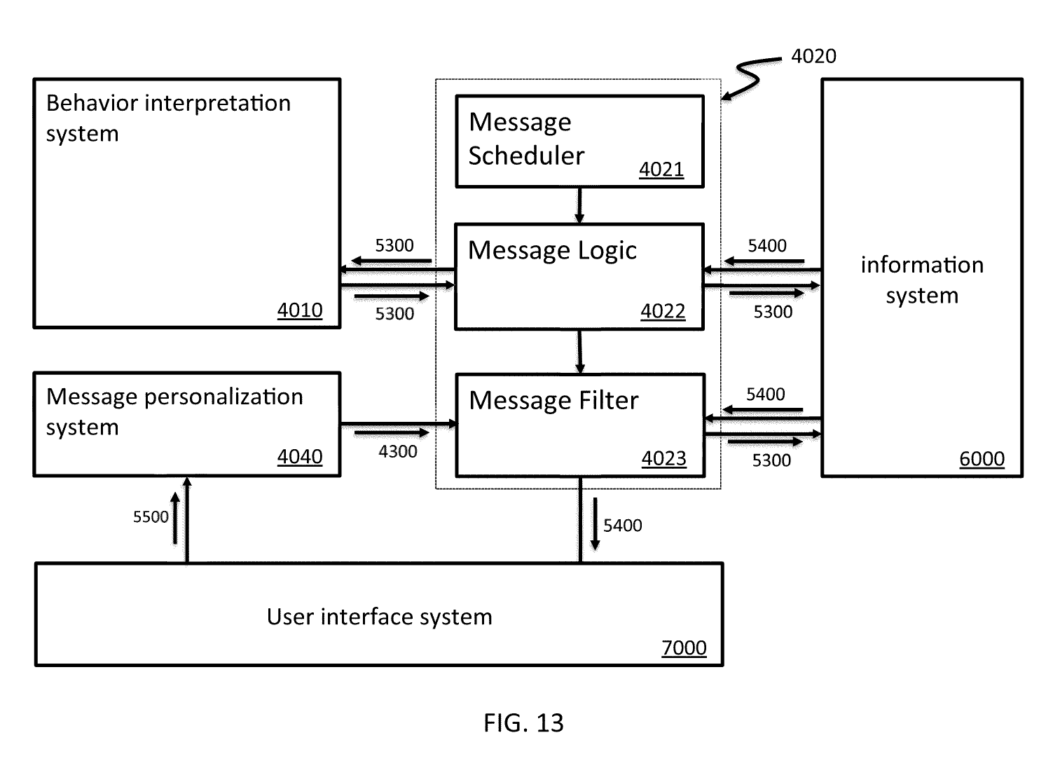

FIG. 13 is a detailed drawing showing yet another alternative embodiment of the message analytics system 4000 of FIG. 12A. As illustrated on FIG. 13, the message generator system 4020 can include a message scheduler 4021, a message logic processor 4022 and a message filter 4023. The message scheduler 4021 can define when to generate the messages 5400. The messages 5400 may be scheduled for delivery at specific predefined times of the day. Alternatively or additionally, the messages 5400 may be scheduled in response to specific user actions, for example, when the user opens the user interface and/or according to specific behaviors analyzed and interpreted by the behavior interpretation system 4010. In another alternative embodiment, the message logic processor 4022 can match the MAs 5300 with the message meta-data stored in the information system 6000. In yet another alternative embodiment, the message filter 4023 can then filter the messages 5400 based on messaging history and the user data 5500, preferably user feedback, and select one or more messages 5400 to be delivered to the user. In yet another alternative embodiment, the message generator system 4020 can also be used to dynamically generate messages 5400 based on the measured behaviors, for instance using semantic inference to automatically generate the messages 5400 related to the message attributes 5300.

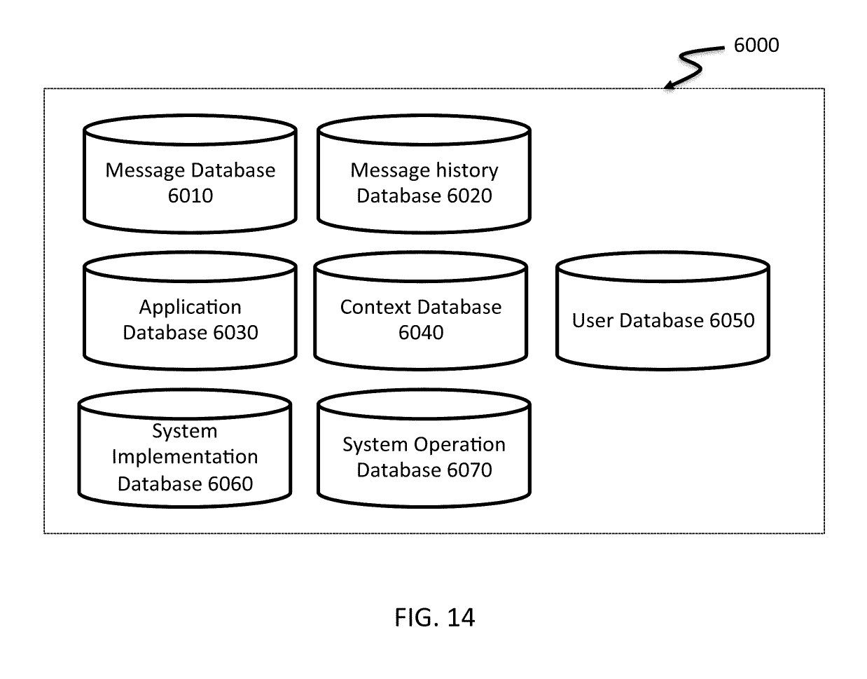

FIG. 14 is a detailed drawing showing an alternative embodiment of the information system 6000. As illustrated on FIG. 14, the information system 6000 stores a portion, or preferably all, of the content of the messaging system 1000. The information system 6000 can include a message database 6010 that stores a portion, or preferably all messages 5400 used by the messaging system 1000. The information system 6000 can also include a message history database 6020. The message history database 6020 can store the messages 5400 that have been delivered to the user during the utilization of the system 1000. The messages 5400 stored in the message history database 6020 can be stored with information on the content of the messages 5400, delivery date, delivery time, etc. The information system 6000 can also include a user database 6050. The user database 6050 can store information about the user. User information may be stored following specific standards such as the Health Insurance Portability and Accountability Act (HIPAA). The information system 6000 can also include an application database 6030, a context database 6040, a system implementation database 6060 and/or a system operation database 6070. The application database 6030 can store information about the application and/or field of use of the system 1000. The context database 6040 can store information about location, time and any other contextual information. The system implementation database 6060 can store information about the implementation of the system 1000. The system operation database 6070 can store information about the operations of the system 1000. In an alternative embodiment, the different pieces of content may be stored in different databases such as the message database 6010, the delivered message database 6020, the application database 6030, the system implementation database 6060, the context database 6040, the user database 6050 and/or the system operation database 6070.

The messages 5400 can be stored in the database under any conventional format, including the format of Message Objects (MOs) that include the message itself and the meta-data about the message. The database can be prepopulated with a list of messages 5400 before the system is deployed. New messages 5400 can be created during the operation of the system. The new messages 5400 can be created by human intervention, or can be generated automatically by the messaging system 1000, for instance by the message generator system 4020.

In another alternative embodiment, the information system 6000 can comprise of multiple message databases 6010 that are field, application and/or user population specific. For instance, the message database 6010 may depend on the ethnicity, culture, gender, age or level of education of the target users.

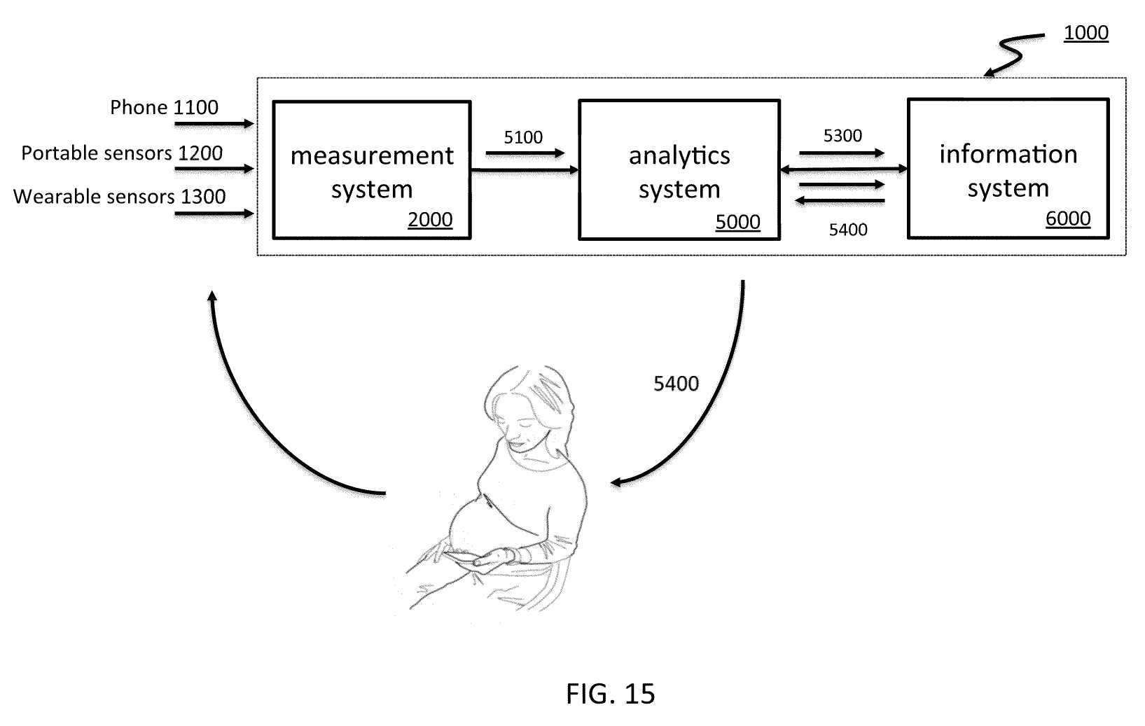

FIG. 15 is a detailed drawing illustrating yet another alternative embodiment of the messaging system 1000. As illustrated on FIG. 15, the messaging system 1000 can be used to motivate positive behaviors and to inspire a healthier lifestyle. Turning to FIG. 15, the measurement system 2000 can use phone sensors 1100, portable sensors 1200 or wearable sensors 1300 to measure raw sensor signals. Raw sensor signals are converted to physical, physiological and/or environment signals 5100 by the measurement system 2000. The signals 5100 are then converted into messages 5400 by the analytics system 5000. The messages 5400 are then stored in the information system 6000, and delivered to the user through the user interface system 7000, for instance through her smart phone.

Data Format

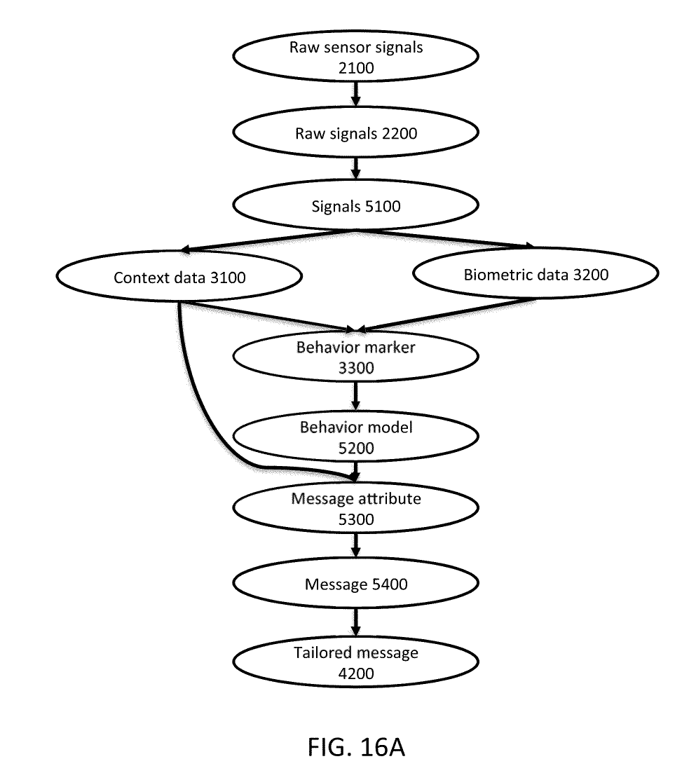

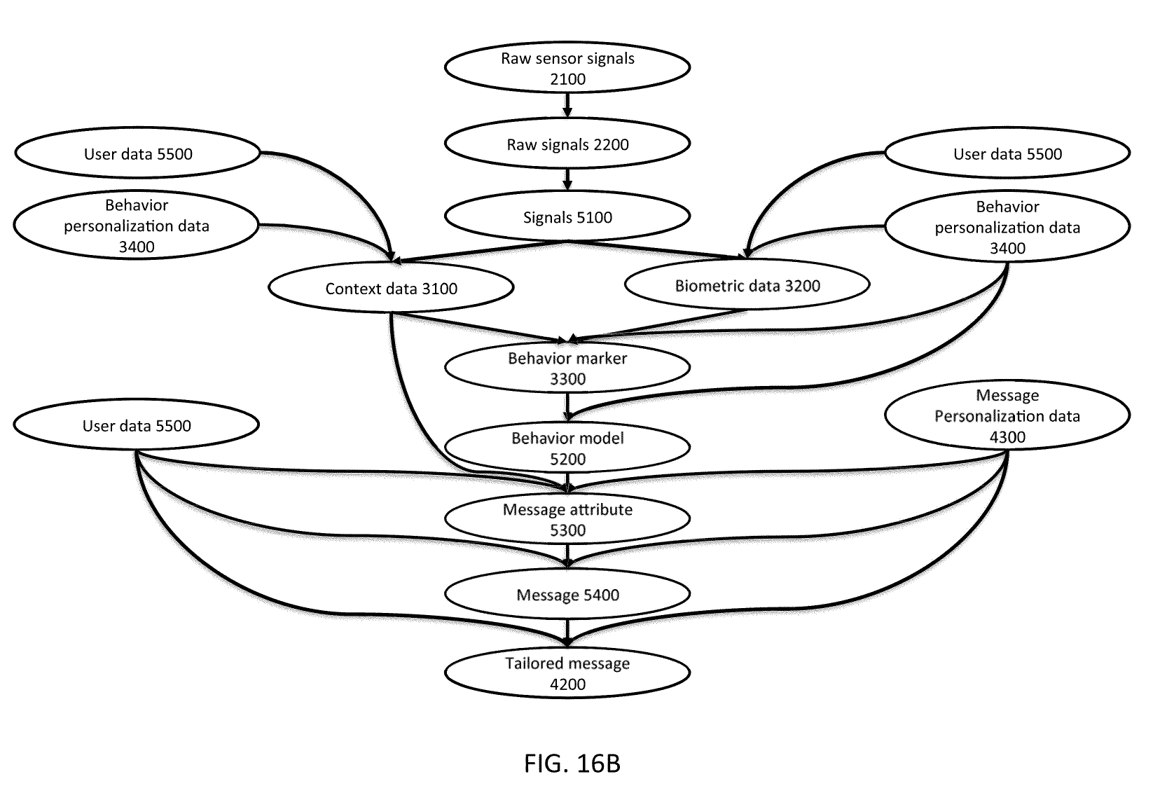

According to the method 100 and system 1000 described above with reference to FIGS. 1-15, the raw sensor signals can undergo a series of transformation to eventually yield a personalized and contextualized message. FIG. 16A is a detailed drawing illustrating a preferred example of how the dataflow can be organized through the messaging system 1000. Turning to FIG. 16A, the data flow can comprise the following:

The raw sensor signals 2100 can be captured by the sensor system 2010 according to receiving raw sensor signals, at 210,

The raw signals 2200 can be outputted by the signal acquisition system 2020 according to acquiring raw sensor signals, at 220,

The physical, physiological and/or environment signals 5100 can be outputted by the signal pre-processing system 2030 according to pre-processing raw signals, at 230,

The contextual data 3100 can be generated by the contextual data processing system 3010 according to processing contextual data, at 320,

The biometric data 3200 can be generated by the biometric data processing system 3020 according to processing biometric data, at 310,

The behavior markers 3300 can be generated by the behavior marker extraction system 3030 according to extracting behavior markers, at 330,

The behavior models 5200 can be generated by the behavior modeling system 3040 according to modeling behavior, at 340,

The message attributes (MAs) 5300 can be generated by the behavior interpretation system 4010 according to interpreting behavior models, at 410,

The messages 5400 can be generated by the message generator system 4020 according to generating messages, at 420,

The tailored messages 4200 can be generated by the message customization system 4030 according to customizing messages, at 430.

FIG. 16B is a detailed drawing illustrating an alternative example of the data flow, wherein the data flow can also include user data 5500, behavior personalization data 3400 and message personalization data 4300. As illustrated on FIG. 16B, user data 5500, behavior personalization data 3400 or message personalization data 4300 can be used for:

processing the signals 5100 to yield the contextual data 3100, at 320,

processing the signals 5100 to yield the biometric data 3200, at 310,

extracting the behavior markers 3300 from the contextual data 3100 and the biometric data 3200, at 330,

modeling behavior based on the behavior markers 3300 to yield the behavior models 5200, at 340,

interpreting the behavior models 5200 to yield the message attributes 5300, at 410,

generating the messages 5400, based on the message attributes 5300, at 420, and/or

customizing the messages 5400 to yield the tailored messages 4200, at 430.

In one preferred embodiment of the method 100 and system 1000 described above with reference to FIGS. 1-15, data objects can be used to organize and structure the data. Multiple data objects can be created, and include, but are not limited to:

Biometric data object

Contextual data object

Behavior marker object

Behavior model object

Message attributes object

Message object (MO)

Tailored message object (TMO)

The biometric data object can contain the biometric data 3200. The contextual data object can include the contextual data 3100. The behavior marker object can include the behavior markers 3300. The biometric data object, the contextual data object or the behavior marker object can take the format of a data point, a data vector, a data matrix, or any combination of points, vectors and matrices. In the context of general health applications, the behavior marker object can contain the following behavior markers 3300: activity, sleep, weight, stress, blood pressure, blood glucose, cortisol, melatonin, cholesterol, etc. Additionally, features can be added that are specific to the field of use or the application. For the field of pregnancy monitoring for instance, relevant additional behavior markers can include: uterine contraction, Braxton-hicks contraction, fetal kicks, fetal movement and fetal stress.

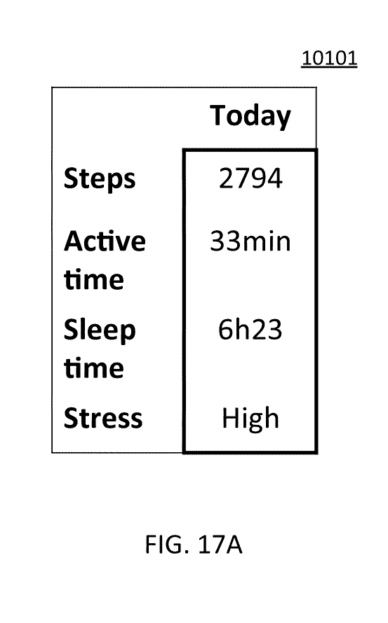

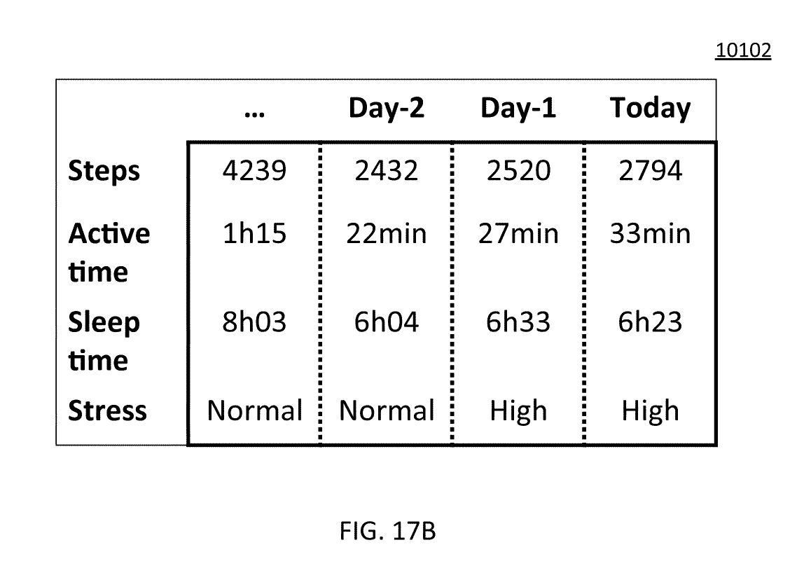

The behavior model object can be a data object that captures the behavior models 5200. FIG. 17A is an exemplary drawing illustrating an embodiment of the behavior model object generated by the behavior analytics system 3000. As illustrated on FIG. 17A, the behavior model can be a vector of selected behavior markers 3300 that represent the user's behavior at a certain point in time.

FIG. 17B is an exemplary drawing illustrating an alternative embodiment of a behavior model object generated by the behavior analytics system 3000. As illustrated on FIG. 17B, the behavior model object can be a matrix of selected behavior markers 3300 that represent the user's behavior a several points in time. For instance, a behavior model object may include behavior markers 3300 for a few successive days.

In another alternative embodiment (not shown), the behavior model object is a sequence, or a pattern, of behavior models 5200 over time. If T is the current moment, then a pattern can comprise the behavior models 5200 at times T, T-1, T-2, etc. Depending on the application, T can have a resolution of seconds, minutes, hours, days, weeks, months, years or any shorter or longer resolution. The choice of a specific resolution depends on the dynamics of the application field in which the method 100 or system 1000 is applied. Consumer health behavior will usually be characterized with a daily model capturing the behavior markers over twenty-four hours, in which case T would have a resolution of one day. In the case of continuous monitoring of vital signs in hospital environment, the health behavior, which, in this context, also refers to the health status of the patient, may change over minutes or hours, and therefore T would have a resolution of seconds or minutes. Certain chronic conditions require managing the chronic behavior on a weekly basis, and for this case T would have a resolution of one week. In some other fields besides healthcare, the behavior modeling process may require very fine time resolution, in the order of seconds, or very coarse resolution in the order of months or even years.

The message attribute object can be a data object that contains the message attributes 5300, representing the interpretation of the behavior models 5200. The message attribute object can contain a set of attributes that are used to generate a message, at 400, or by the message generator system 4020, and can take into account contextual data 3100, target behavior 4100 and user data 5500. In one embodiment, the message attributes 5300 correspond to selected fields of the message meta-data.

The message object (MO) can be composed of the message 5400 and message meta-data. The message meta-data can describe and/or characterize each message 5400. The message meta-data can be used by the message generator system 4020 to generate messages 5400 that match the message attributes 5300. The message attributes 5300 can be defined and/or modified by the behavior interpretation system 4010. The message attributes 5300 can be defined and/or modified by the message scheduler 4021 to select the best time to deliver the message. The message attributes 5300 can be defined and/or modified by the user interface system 7000 to display the messages 5400 in proper way. The meta-data is uniquely associated to each message 5400. The message 5400 and message meta-data are stored in the message object. The fields of the meta-data can be modified throughout the message generation process according to method 100, and by any systems composing the messaging system 1000.

The tailored message object is a customized version of the message object, in which the text of the message 5400 or the message meta-data has been advantageously modified by the message customization system 4030 to reflect personal preferences of the user.

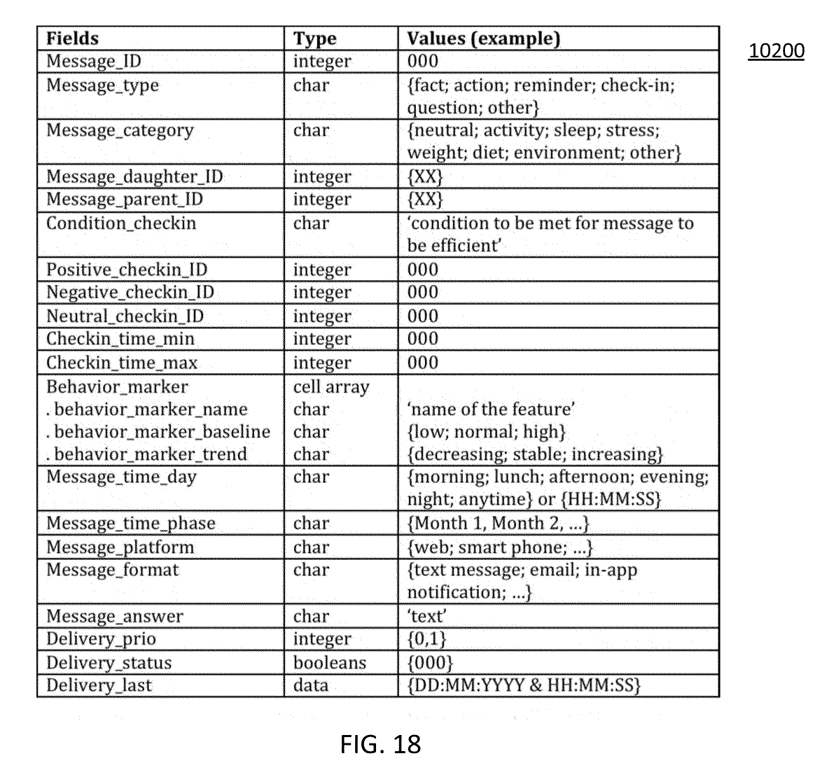

Message meta-data can be structured in any conventional ways. FIG. 18 is a detailed drawing illustrating a preferred embodiment of message meta-data, for which example values are provided for the different fields:

Message_ID: the unique identifier of the message

Message_type: the type of the message; the message can be a fact, an action, a reminder, a check-in, a question, or other.

Message_category: the category of the message, referring to what aspect of behavior or health it is associated to. For example the message can be associated to activity, sleep, stress, weight, diet, environment or to no specific behavior aspect (neutral).

Message_daughter_ID: the unique identifier of the next message that should logically follow the current message (if applicable)

Message_parent_ID: the unique identifier of the message that logically precede the current message (if applicable)

Condition_checkin: the condition to be met for the message to be considered efficient

Met_checkin_ID: the unique identifier of the check-in that should be delivered to the user in case the expected effect of the current message is met

Unmet_checkin_ID: the unique identifier of the check-in that should be delivered to the user in case the expected effect of the current message is unmet

Neutral_checkin_ID: the unique identifier of the check-in that should be delivered to the user in case the current message did not have any effect

Checkin_time_min: the minimum time after which a check-in may be pushed

Checkin_time_max: the maximum time before which a check-in may be pushed

Behavior_marker: a array of behavior markers relevant to the message, with the following information associated to each marker:

Behavior_marker_name: name of the behavior market

Behavior_marker_baseline: the baseline of the behavior marker

Behavior_marker_trend: the trend of the behavior marker

Message_time_day: the time of day at which the message should be delivered.

Message_time_phase: the phase of the behavior change program at which the message should be delivered. For instance, in a 6 months behavior change program, a message may be appropriate only for the first two months.

Message_platform: the platform on which the message is preferably delivered.

Message_format: the format in which the message is preferably delivered.

Message_answer: possible answers or feedback information that the user may generate in response to this message

Delivery_prio: the priority at which the message should be delivered

Delivery_status: the status of the current message made of at least 3 boolean variable; for instance {read, in-queue, deleted}

Delivery_last: last date and time at which the message was delivered to the user

Meta-data can be used in multiple ways. In a preferred embodiment (not shown), the message attributes 5300 generated by the behavior analytics system 3000 can be compared with the meta-data of the messages 5400 in the message database 6010. The messages 5400 for which the meta-data match the message attributes 5300 are selected as possible messages 5400 to be pushed to the user. For example, the message attributes 5300 generated by the behavior interpretation system 4010 and composing the message attributes 5300 may include: message_type, message_category, message_time_day, message_time_phase, behavior_marker, behavior_marker_baseline, behavior_marker_trend, delivery_prio.

Recap & Implementation

The method 100 and system 1000 described above with reference to FIGS. 1-15 enable biometric and context based messaging and can prove desirable for a wide range of applications, such as wellness, fitness, health and healthcare, or to motivate healthy habits or behaviors.

The generic and sophisticated method 100 advantageously generates personalized messages 5400 based on sensor data, also called raw sensor signals, 2100. The method 100 has multiple advantages. Some of them are discussed below.

First, the method 100 and system 1000 can enable the messages 5400 to be delivered at a time that is based on the user's behavior as measured by the system 1000, on the type of the messages 5400 being delivered and/or on the user data 5500. Preferably, the user data 5500 can include user feedback. Accordingly, the method 100 and system 1000 can enable the delivery of a message at the right time. For example, the right time can be the most effective time for triggering an action that will help motivating behavior change.

Furthermore, the method 100 and system 1000 can use contextual data 3100 to gather information about the environment and location of the user. Accordingly, the method 100 and system 1000 can deliver the messages 5400 at the right place. For example, the right place can be the place that will maximize its impact on the target behavior change.

Furthermore, the method 100 and system 1000 can generate messages 5400 that are directly related and triggered by the user's behavior as inferred from biometric data 3200 and contextual data 3100. Accordingly, the method 100 and system 1000 can provide the messages 5400 that are directly relevant to the user and to her recent activity, thus improving the impact of the messages 5400.

Furthermore, the method 100 and system 1000 can provide multiple levels of personalization. Personalization can be achieved according to any of the following ways:

personalization of the behavior analytics, at 350,

personalization of the message analytics, at 440,

customization of the message delivered to the user, at 430, and/or

specific scheduling of the message delivery, at 421.

According to the method 100 and system 1000, the content of the messages 5400 can be advantageously personalized using the user's specific behavior at a certain point in time. The messages 5400 can be further customized in tone, format and content based on user's preferences and user's feedback. The customization can happen dynamically as the user uses the system, and the system learns the user's behaviors, habits and preferences. Accordingly, the method 100 and system 1000 can provide personalized and adaptive messaging to the user.

An aspect of effective messaging is the ability to adapt to personal information regarding the user, and to individual preferences of the user.

In one embodiment, personalization of the behavior analytics, at 350, personalization of the message analytics, at 440, message customization, at 430 or scheduling messages, at 421, is achieved using data extracted from the sensors collected by the measurement system 2000. In a further embodiment, personalization of the behavior analytics, at 350, personalization of the message analytics, at 440, message customization, at 430 or scheduling messages, at 421, is achieved using information entered by the user, for instance through the user interface system 7000.

By generating the messages 5400 based on the behavior models 5200 derived from the biometric data 3200 and contextual data 3100, a higher and more complete level of messaging can be achieved. According to the method 100 and system 1000, the level of messaging can be elevated by combining multiple sensors, combining multiple aspects of behaviors and/or combining patterns of behaviors over time.

In further embodiments of the system 1000, the measurement system 2000, the analytics system 5000 and the information system 6000 can be arranged and provided for performing the method 100 as disclosed above.

The method 100 described above with reference to FIGS. 1-8C may be practiced in hardware, in software and/or a combination of hardware and software. To this end, the method 100 also relates to a computer program and a computer program product comprising program code means, which computer program functions to carry out the method 100, when the computer program is loaded in a working memory of a computer and is executed by the computer.

Examples of Messaging Analytics

To illustrate the messaging analytics 4000, we consider the following examples. For all examples that follow, and for sake of clarity, let it be that the behavior analytics system 3000 has generated a behavior model 5200 such as the one illustrated in FIG. 17B, in which each column corresponds to a day, and each row corresponds to a behavior marker 3300. Let it also be that the message generator system 4020 triggers the generation of a message 5400 based on the detection of a certain event. For instance the event may be the fact that the user is interacting with the system 1000 through the user interface 7000, or the detection of a specific behavior or behavior pattern. In one embodiment, the message generator system 4020 can directly trigger the behavior interpretation system 4010 to analyze the behavior model 5200. The behavior interpretation system 4010 can consist of a decision tree that analyzes the last set of behavior markers 3300. The decision tree may consist in a set of sequential conditional statements applied to each behavior marker 3300 of the behavior model 5200.

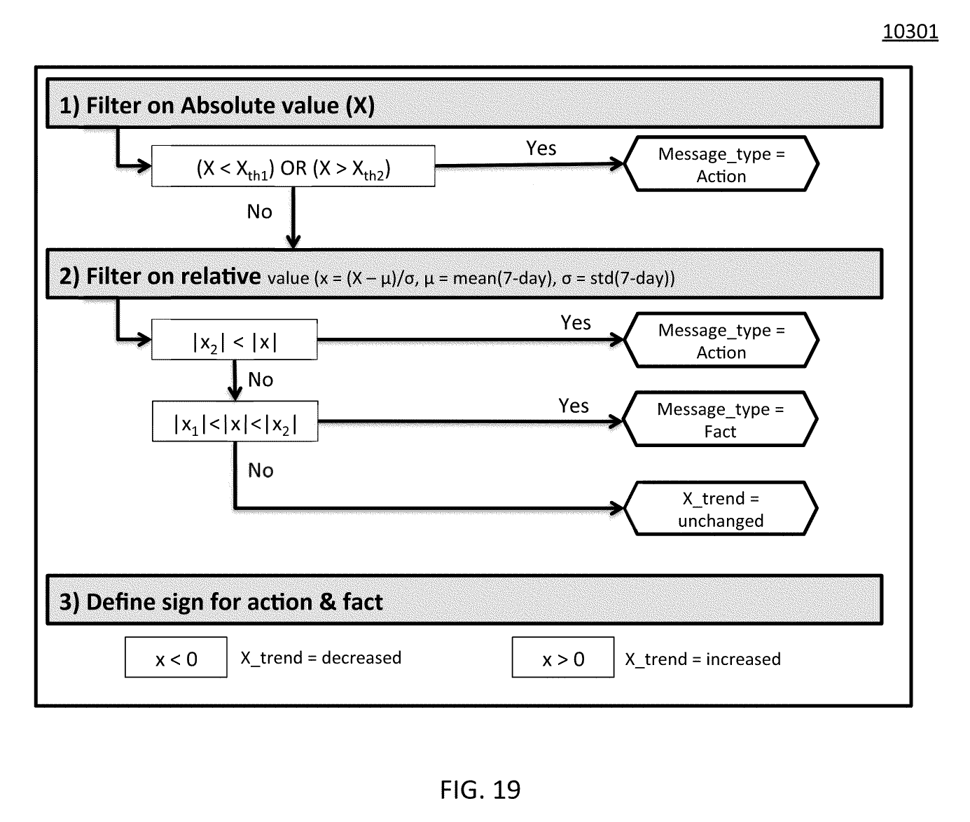

FIG. 19 is an exemplary drawing illustrating one embodiment of the message analytics system 4000. As illustrated on FIG. 19, the behavior interpretation system 4010 can operate as the decision tree 10301. First the absolute value of a behavior marker 3300, labeled X for the sake of this example, is compared with a maximum and a minimum threshold. If the absolute value of the behavior marker X is greater than the maximum threshold, or smaller than the minimum threshold, then the message_type field of the message attribute 5300 is set to ‘action’. If not, the behavior interpretation system 4010 looks at the relative deviation of the behavior marker X compared to its baseline. Two thresholds are defined (x₁ and x₂). If the relative deviation is higher than the second threshold, then the message_type field of the message attribute 5300 is set to ‘action’. If the relative deviation is between the first and the second threshold, then the message_type field of the message attribute 5300 is set to ‘fact’. If the relative deviation is smaller than the first threshold, nothing happens. Finally, the trend of the behavior marker X is defined based on the sign of the relative deviation, and the behavior_marker_trend field of the message attribute 5300 is set accordingly. The message attributes 5300, including the updated message_type and the behavior_marker_trend, are then passed to the message generator system 4020. The message generator system 4020 then queries the information system 6000 for messages 5400 of the right type. The result is a set of messages 5400 that can be delivered to the user. The message generator system 4020 then selects one message 5400 based on the timing and on the history of messages 5400, and delivers it to the user in due time.

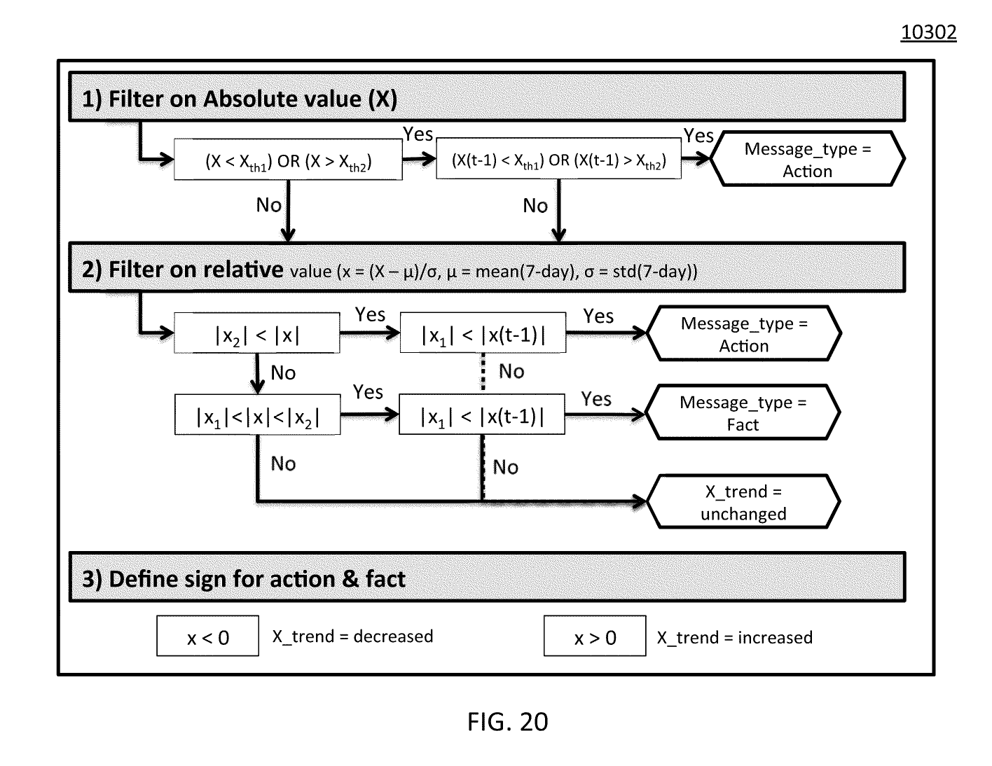

FIG. 20 is an exemplary drawing illustrating an alternative embodiment of the message analytics system 4000. As illustrated on FIG. 20, the behavior interpretation system 4010 can operate as the decision tree 10302, wherein the decision tree 10302 can use the last two values of each behavior marker 3300. The principle of the decision tree 10302 is similar to the decision tree 10301, to the exception that the information about the last two days is used in the conditional logic to define the value of the fields of the message attributes 5300, and eventually to generate the messages 5400.

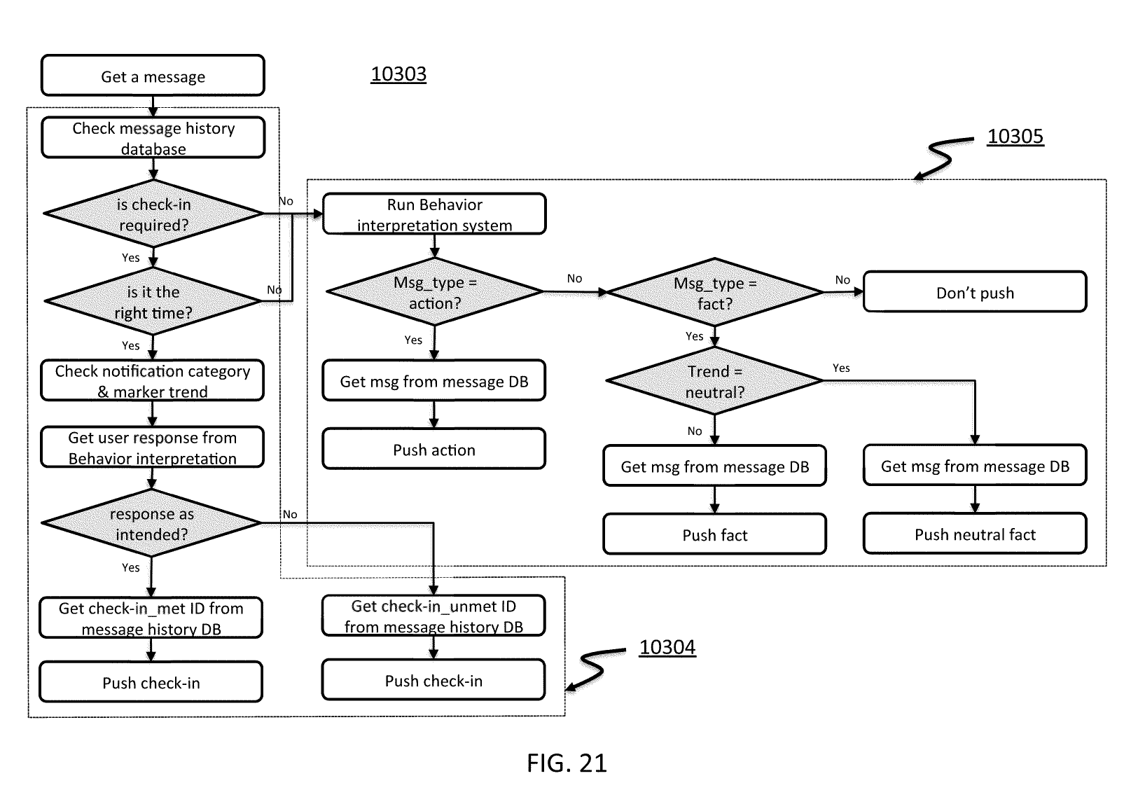

FIG. 21 is an exemplary drawing illustrating another alternative embodiment of the message analytics system 4000. As illustrated on FIG. 21, the message generator system 4020 can include more elaborated logic 10303 to decide between different types of messages 5400, for example check-in, action or fact. As illustrated on FIG. 21, when the message generator system 4020 triggers the generation of a message 5400, the message generator system 4020 first enters the check-in logic 10305 to check if a message of type ‘check-in’ should be delivered. First the message generator system 4020 queries the information system 6000, for instance the message history database 6020, to check if any messages 5400 delivered in the past requires a check-in. If one of the past messages 5400 requires a check-in, then the message generator system 4020 further checks if it is the right time to deliver the message. If the time is right, then the message generator system 4020 calls the behavior interpretation system 4010 to analyze the behavior marker trend, in comparison to the expected evolution that should be triggered by the past message, and defines the type of check-in accordingly. For example, check-in may take two values: ‘met’ or ‘unmet’. If a check-in needs to be delivered, the process stops here and the message generator system 4020 queries the information system 6000, in particular the message database 6010, for a message 5400 of the appropriate type. If no check-in needs to be delivered, the process continues and the message generator system 4020 enters the notification logic 10305 and calls the behavior interpretation system 4010 to analyze the behavior model 5200, for example using the decision tree 10301 or the decision tree 10302.

As illustrated in FIG. 21, the message analytics system 4000 can include several levels of logic, for example check-in logic 10304 and notification logic 10305, to generate the message 5400.

The above examples illustrate how the messaging analytics system 4000 can operate, but do not provide an exhaustive description of the possible implementations for the messaging analytics system 4000. It will be clear for the one skilled in the art that the number of possibilities for implementing messaging analytics system 4000 is numerous.

### Example

### Motivating a Balanced Lifestyle

To illustrate the method 100 and the system 1000 for biometric and context based messaging, we consider one embodiment of the system 1000 designed to drive behavior change towards a balanced lifestyle. For the sake of this example, a balanced lifestyle is defined as a way of living that balances activity, sleep and stress. In this example, the messaging system 1000 is implemented as a smartphone software application.

The measurement system 2000 comprises of the accelerometer sensor, the camera sensor and the LED embedded in a smartphone, measuring physical (movement) and physiological (photoplethysmograph or PPG) signals. The accelerometer signal is monitored continuously. The PPG signal is recorded at least once per day, in a short routine during which the user is covering the camera with her finger for the PPG to be measured. Cell phone usage is also monitored.

The biometric data processing system 3020 and behavior marker extraction system 3030 process the accelerometer and PPG signals to extract the number of steps (from the accelerometer), heart rate and heart rate variability (from PPG). From the cell phone usage the contextual data processing system 3010 extracts the last time at which the user used the app.

The behavior modeling system 3040 uses the number of steps, heart rate, heart rate variability and app usage to infer the behavior of the user. The behavior model 5200 is defined by the following behavior markers 3300: the number of steps, the active time, the sleep time, and the stress level. The number of steps comes directly from the biometric data processing system 3020. The active time is computed by the behavior market extraction system 3030, based on the step count. The sleep time is computed by the behavior market extraction system 3030, based on the step count and the last time the user used the app. The stress level is computed by the behavior market extraction system 3030, from heart rate variability measured during the daily routine. A new state of the behavior model 3200 is defined every day, at the end of the day. The behavior model 5200 is a matrix which rows correspond to the behavior markers 3300, and which columns correspond to the day. For today the behavior model 5200 is denoted BM(T). The last column contains the behavior markers 3300 for today, the second last contains the behavior markers 3300 for yesterday, and so on.

The message analytics system 4000 takes as input the behavior models 5200 for the last week, the target behavior change (balanced lifestyle) 4100 and the user data 5500 to generate the messages 5400. The behavior interpretation system 4010 analyses the behavior model 5200 and generates the message attributes 5300 that are used to select a specific message 5400 from the message database 6010. The message attributes 5300 used in this example are activity_baseline, activity_trend, sleep_baseline, sleep_trend, stress_baseline, stress_trend. The activity_baseline, sleep_baseline and stress_baseline attributes can be ‘low’, ‘neutral’ or ‘high’, based on the average value of the behavior marker 3300 over the last 7 days. The activity_trend, sleep_trend and stress_trend attributes can be ‘decreased’, ‘unchanged’ or ‘increased’. A decision tree is used to calculate the value of the message_type attribute, for example using the process described in FIG. 19, FIG. 20 or FIG. 21. The message generator system 4020 then identifies the messages 5400 in the message database 6010 for which the meta-data match the message attributes 5300, and select one message 5400. The message 5400 can be customized or edited by the messaging customization system 4030 based on user data 5500. User data 5500 can for example include the feedback gathered from the user on previous messages 5400. Finally the message scheduler 4012 defines a time at which the message 5400 should be delivered to the user.

The user interface system 7000 is part of the smartphone software application. The message 5400 is delivered as a notification in the application, and/or as a pushed notification. The user can like or delete the notification. The information about liking or deleting is stored as part of the user data 5500. The user data 5500 can be used by the behavior personalization system 3050 and/or the message personalization system 4040. The user data 5500 is also stored in the information system 6000, preferably in the user database 6050.

In an alternative embodiment of the messaging system 1000, GPS is measured by the measurement system 2000 and processed by the contextual data processing system 3010, to provide contextual data 3100 regarding the location of the user.

In another alternative embodiment of the messaging system 1000, the measurement system 2000 can include a wearable sensor, for example a chest-strap heart rate monitor or a wrist-based pulse measurement system. The wearable sensor can be used to measure heart rate and heart rate variability.

In yet another alternative embodiment of the messaging system 1000, the measurement system 2000 can include a weight scale to measure the weight of the user. The weight can be used by the behavior analytics system 3000 to yield the behavior model 5200.

In yet another alternative embodiment of the messaging system 1000, the measurement system 2000 can include a blood pressure monitor to track blood pressure over time. The blood pressure can be used by the behavior analytics system 3000 to yield the behavior model 5200.

In yet another alternative embodiment of the messaging system 1000, a blood glucose monitor is used to track blood glucose levels over time. The blood glucose can be used by the behavior analytics system 3000 to yield the behavior model 5200.

### Example

### Motivating Healthy Pregnancy

The above example of the balanced lifestyle is applicable to any individual. It is however particularly relevant for pregnant woman since achieving a balanced lifestyle is not only important for her health, but also for the health of her future baby. The messaging system 1000 as described in the previous example can directly be applied to the case of pregnancy monitoring. In one alternative embodiment of messaging system 1000, the information system 6000, and in particular the message database 6010, may be adapted to include pregnancy related messages 5400.

In one alternative embodiment of messaging system 1000, the type of messages 5400 is adapted depending on the ethnicity, culture and level of education of the target users.

In one alternative embodiment of messaging system 1000, the user interface system 7000 is used to log information about contraction.

In one alternative embodiment of messaging system 1000, the measurement system 2000 can include a wearable sensor to track or differentiate Braxton-Hicks and real contractions. The contraction can be used by the behavior analytics system 3000 to yield the behavior model 5200.

In one alternative embodiment of messaging system 1000, the user interface 7000 is used to log information about fetal activity and kicks.

In one alternative embodiment of messaging system 1000, the measurement system 2000 can include a wearable sensor to track fetal activity and kicks. The fetal activity and kicks can be used by the behavior analytics system 3000 to yield the behavior model 5200.

The disclosed embodiments are susceptible to various modifications and alternative forms, and specific examples thereof have been shown by way of example in the drawings and are herein described in detail. It should be understood, however, that the disclosed embodiments are not to be limited to the particular forms or methods disclosed, but to the contrary, the disclosed embodiments are to cover all modifications, equivalents, and alternatives.

What is claimed is:

1. A method for generating a personalized message for motivating behavior change based on biometric and contextual data, comprising:
receiving at least one of physical data, physiological data and environmental data from a sensor associated with a user device;
extracting the biometric data and contextual data from the received data;
analyzing selected biometric data with relevant contextual data to form respective behavior markers;
generating a behavior model from the behavior markers;
converting the behavior model into a plurality of message attributes;
creating the personalized message based upon the plurality of message attributes; and
delivering the personalized message to the user device.

2. The method of claim 1, wherein said extracting the biometric data and the contextual data comprises one of:
extracting the biometric data from the received physical or physiological data and extracting the contextual data from the received physical or environmental data based upon the extracted biometric data; and
extracting the contextual data from the received physical or environmental data and extracting the biometric data from the received physical or physiological data based upon the extracted contextual data.

3. The method of claim 1, wherein at least one of said extracting the biometric data and the contextual data from the received data, said combining the selected biometric data with the relevant contextual data, and said generating the behavior model from the behavior markers is personalized based upon a change in the contextual data over time.

4. The method of claim 1, wherein at least one of said extracting the biometric data and the contextual data from the received data, said combining the selected biometric data with the relevant contextual data, and said generating the behavior model from the behavior markers is personalized based upon a change over time in user data provided via the user device.

5. The method of claim 1, wherein said converting the behavior model includes specifying a time at which the personalized message should be delivered to the user device.

6. The method of claim 5, wherein said specifying the time at which the personalized message should be delivered to the user device includes scheduling the personalized message for delivery to the user device based upon at least one of daily routine data, location data, and a predetermined user action being performed on the user device.

7. The method of claim 1, wherein said creating the personalized message includes scheduling the personalized message for delivery to the user device.

8. The method of claim 7, wherein said creating the personalized message comprises dynamically creating the personalized message based upon the plurality of message attributes.

9. The method of claim 7, wherein said creating the personalized message further comprises providing the plurality of message attributes to a database system, selecting one or more messages from among a plurality of messages available from the database system based upon the plurality of message attributes, reading the selected messages from the database system, and filtering the selected messages to identify the personalized message.

10. The method of claim 9, further comprising storing metadata for uniquely characterizing each of the plurality of messages in the database system, wherein said selecting the one or more messages includes comparing the metadata for the plurality of messages with the plurality of message attributes.

11. The method of claim 1, wherein said creating the personalized message includes customizing the personalized message based upon user data provided via the user device.

12. The method of claim 11, wherein said creating the personalized message includes at least one of customizing the personalized message based upon a change in the user data over time and filtering the personalized message based upon at least one of messaging history, the user data, and user feedback.

13. A computer program product for generating a personalized message for motivating behavior change based on selected biometric and related contextual data, the computer program product being encoded on non-transitory machine-readable storage media and comprising:
instruction for receiving at least one of physical data, physiological data and environmental data from a sensor associated with a user device;
instruction for extracting biometric data and contextual data from the received data;
instruction for selecting the selected biometric data from the extracted biometric data;
instruction for identifying the related contextual data being relevant to the selected biometric data;
instruction for processing the selected biometric data with the related contextual data to form respective behavior markers;
instruction for generating a behavior model from the behavior markers;
instruction for converting the behavior model into a plurality of message attributes;
instruction for creating the personalized message based upon the plurality of message attributes; and
instruction for delivering the personalized message to the user device.

14. The computer program product of claim 13, wherein said instruction for creating the personalized message includes instruction for providing the plurality of message attributes to a database system, instruction for selecting one or more messages from among a plurality of messages available from the database system based upon the plurality of message attributes, instruction for reading the selected messages from the database system, and instruction for filtering the selected messages to identify the personalized message.

15. The computer program product of claim 14, further comprising instruction for storing metadata for uniquely characterizing each of the plurality of messages in the database system, wherein said instruction for selecting the one or more messages includes instruction for comparing the metadata for the plurality of messages with the plurality of message attributes.

16. A system for generating a personalized message for motivating behavior change based on biometric and contextual data, comprising:
a user device associated with a sensor; and
a messaging system, comprising:
a measurement system for receiving at least one of physical data, physiological data and environmental data from the sensor associated with the user device;
a behavior analytics system for extracting the biometric data and contextual data from the received data, analyzing selected biometric data with relevant contextual data to form respective behavior markers, generating a behavior model from the behavior markers; and
a messaging analytics system for converting the behavior model into a plurality of message attributes, creating the personalized message based upon the plurality of message attributes, and delivering the personalized message to the user device.

17. The system of claim 16, wherein said user device is a smartphone, and wherein said sensor is selected from the group consisting of an accelerometer, a motion sensor, a Global Positioning System device, a camera, a light emitting diode, and an optical sensor embedded in said smartphone.

18. The system of claim 16, wherein said sensor includes at least one wearable sensor for providing at least one of the physical data, the physiological data and the environmental data on a continuous basis.

19. The system of claim 16, wherein said messaging analytics system selects one or more messages from among a plurality of messages available from a database system based upon the plurality of message attributes, reads the selected messages from the database system, and filters the selected messages to identify the personalized message.

20. The system of claim 16, further comprising an information system for storing a plurality of messages and metadata for uniquely characterizing each of the plurality of messages, wherein said messaging analytics system creates the personalized message by comparing the metadata for the plurality of messages with the plurality of message attributes.
